(19) United States
(12) Patent Application Publication
(10) Pub. No.: US 2023/0207063 A1
(43) Pub. Date: Jun. 29, 2023

(54) DYNAMIC GRAPHICAL STATUS SUMMARIES FOR NUCELOTIDE SEQUENCING

(71) Applicants: Illumina Software, Inc., San Diego, CA (US); Illumina Cambridge Limited, Cambridge (UK)

(72) Inventors: Jeremy Ward, Escondido, CA (US); Rodger Constandse, San Diego, CA (US); Michael Smoot, San Diego, CA (US); Michelle Beoris, San Diego, CA (US); Adam French, Cambridge (GB)

(21) Appl. No.: 17/936,722

(22) Filed: Sep. 29, 2022

Related U.S. Application Data
(60) Provisional application No. 63/293,562, filed on Dec. 23, 2021.

(51) Int. Cl. G16B 30/20; H04L 67/06

(52) U.S. Cl. CPC G16B 30/20; H04L 67/06

(57) ABSTRACT

This disclosure describes methods, non-transitory computer readable media, and systems that can query the status of various stages in an end-to-end sequencing process and generate a graphical status summary for the sequencing process that depicts icons indicating statuses of the various stages. For instance, the disclosed systems can generate a graphical status summary for a nucleotide sequencing taskset that includes icons depicting statuses of a sequencing run, a data transfer of base-call data to a device for variant analysis, and the variant analysis—each part of the same nucleotide sequencing taskset. By exchanging data with a sequencing device for read data and one or more servers for variant analysis, the disclosed system can quickly provide a graphical status summary of an end-to-end sequencing process marked by various tasks within a nucleotide sequencing taskset.

### CROSS-REFERENCE TO RELATED APPLICATIONS

The present application claims the benefit of, and priority to, U.S. Provisional Application No. 63/293,562, entitled “DYNAMIC GRAPHICAL STATUS SUMMARIES FOR NUCLEOTIDE SEQUENCING,” filed on Dec. 23, 2021. The aforementioned application is hereby incorporated by reference in its entirety.

### BACKGROUND

In recent years, biotechnology firms and research institutions have improved hardware and software for both sequencing machines that determine nucleotide-fragment reads for a sample genome (or other nucleic-acid polymer) and sequencing-data-analysis software that analyze the base calls for such nucleotide-fragment reads. To facilitate managing sequencing, some existing sequencing machines include on-machine software that cause the sequencing machines to display interactive graphical options to start and stop sequencings runs. After sequencing runs finish, such on-machine software can likewise cause the sequencing machines to display options to view detailed sequencing metrics for respective sequencing runs. In addition to on-machine software, existing sequencing-data-analysis software often run on separate computing devices to align base-call data for nucleotide-fragment reads with a reference genome and determine (and depict) data for variants of nucleotide bases that differ from the reference genome. While such on-machine software and sequencing-data-analysis software (together “existing sequencing management systems”) provide useful options to start and stop sequencing runs or view results of sequencing-data analysis, existing sequencing management systems (i) generate limited or misleading graphic grids depicting separate sequencing machine processes and separate sequencing-data-analysis processes and (ii) limit functions and control of an end-to-end sequencing process for a sample across the sequencing machine and sequencing-data analysis.

From a wholistic viewpoint, for example, existing sequencing management systems often provide graphical user interfaces with a limited or misleading snapshot of the end-to-end sequencing process for genome samples. To illustrate, some existing systems offer graphical user interfaces with only partial information concerning samples' sequencing process and corresponding analysis, but omit critical information concerning such a process and analysis. Similarly, some existing systems offer only separate graphical user interfaces that depict aspects of a sequencing run on one computing device and aspects of corresponding variant information on a separate computing device. But these existing and separate graphical user interfaces isolate information concerning a sequencing run and the sequencing-data analysis for variants—thereby obfuscating the big picture and limiting control over the end-to-end sequencing process.

In addition to graphical user interfaces with isolated graphical information and limited functionality, existing sequencing management systems provide misleading graphics that omit critical information concerning the end-to-end sequencing process for genome samples or hide such critical information behind layers of different graphical user interfaces. For instance, in some existing graphic grids, existing systems provide generalized graphics concerning a sequencing run and a corresponding secondary-data analysis for variants. But conventional sequencing graphics fail to indicate when an error or breakdown has occurred at certain points after the sequencing run or after the corresponding sequencing-data analysis. By failing to indicate such errors, existing systems often offer no visibility into a sequencing process with multiple potential failure points.

Independent of graphic grids that omit or obfuscate critical information, existing sequencing management systems often generate graphical user interfaces with either no corresponding functionalities or selectable options of limited functionality. To access certain tools relevant to a sequencing run or a corresponding sequencing-data analysis for variants, for instance, existing systems require users to either navigate across multiple graphical user interfaces for a particular stage in between the sequencing run and post-sequencing-data-analysis stages or initiate a graphical user interface from an entirely different software program.

These, along with additional problems and issues exist in existing sequencing management systems.

### SUMMARY

This disclosure describes one or more embodiments of systems, methods, and non-transitory computer readable storage media that solve one or more of the problems described above or provide other advantages over the art. In particular, the disclosed system can query the status of various stages in an end-to-end sequencing process and generate a graphical status summary for the sequencing process that depicts icons indicating statuses of the various stages. For instance, the disclosed systems can generate a graphical status summary for a nucleotide sequencing taskset that includes icons depicting statuses of a sequencing run, a data transfer of base-call data to a device for variant analysis, and the variant analysis—each part of the same nucleotide sequencing taskset. By exchanging data with a sequencing device for read data and one or more servers for variant analysis, the disclosed system can quickly provide a graphical status summary of an end-to-end sequencing process marked by various tasks within a nucleotide sequencing taskset. From a graphical user interface depicting graphical status summaries for active nucleotide sequencing tasksets, the disclosed system can also cause computing devices to display selectable options for viewing more detailed summaries of individual stages or for intervening at particular stages of a given nucleotide sequencing taskset.

Additional features and advantages of one or more embodiments of the present disclosure will be set forth in the description which follows, and in part will be obvious from the description, or may be learned by the practice of such example embodiments.

### BRIEF DESCRIPTION OF THE DRAWINGS

The detailed description refers to the drawings briefly described below.

FIG. 1 illustrates an environment in which a sequencing status system can operate in accordance with one or more embodiments of the present disclosure.

FIG. 2 illustrates a schematic diagram of the sequencing status system querying the status of various nucleotide sequencing tasks part of a nucleotide sequencing taskset and generating a graphical status summary for the nucleotide sequencing taskset in accordance with one or more embodiments of the present disclosure.

FIG. 3A illustrates a graphical user interface for planned nucleotide sequencing tasksets for respective nucleotide sequencing tasksets in accordance with one or more embodiments of the present disclosure.

FIGS. 3B-3F illustrate graphical user interfaces comprising graphical status summaries for respective nucleotide sequencing tasksets in accordance with one or more embodiments of the present disclosure.

FIG. 3G illustrates a graphical user interface for completed nucleotide sequencing tasksets in accordance with one or more embodiments of the present disclosure.

FIGS. 4A-4B illustrates graphical user interfaces comprising detailed summaries for respective nucleotide sequencing tasksets in accordance with one or more embodiments of the present disclosure.

FIG. 5 illustrates an example architecture diagram of the sequencing status system in relation to an overall sequencing environment in accordance with one or more embodiments.

FIGS. 6A-6B illustrate series of acts for querying the status of various tasks of a nucleotide sequencing taskset and generating a graphical status summary for the nucleotide sequencing taskset in accordance with one or more embodiments of the present disclosure.

FIG. 7 illustrates a block diagram of an example computing device in accordance with one or more embodiments of the present disclosure.

FIG. 8 illustrates a block diagram of an example optical system for image-based genomic sequencing in accordance with one or more embodiments.

FIG. 9 illustrates an example imager for image-based genomic sequencing in accordance with one or more embodiments.

FIG. 10 illustrates an example diagram for performing image-based genomic sequencing in accordance with one or more embodiments.

### DETAILED DESCRIPTION

This disclosure describes one or more embodiments of a sequencing status system that queries the status of various nucleotide sequencing tasks within a taskset and generates a graphical status summary that efficiently depicts the status of individual tasks from the nucleotide sequencing taskset. For instance, the sequencing status system can send or receive data with a sequencing device concerning a sequencing run status and also determine statuses (e.g., from other computing devices) for various other nucleotide sequencing tasks for variant analysis following the sequencing run. Based on real time status queries, the sequencing status system generates a graphical status summary for a nucleotide sequencing taskset that includes a grouped series of dynamic status icons depicting statuses of various tasks, such as a sequencing run, a data transfer of base-call data to a device for variant analysis, and the variant analysis. As computing devices progress from executing a sequencing run to nucleotide sequencing tasks associated with a corresponding variant analysis for different nucleotide sequencing tasksets, the sequencing status system can detect a corresponding status and change the representative icon to efficiently communicate the status of both critical nucleotide sequencing tasks and an overall progress of an active nucleotide sequencing taskset.

To illustrate, in one or more embodiments, the sequencing status system receives a status query from a computing device concerning a nucleotide sequencing taskset for determining sample genomes' composition using a sequencing device and a server for variant analysis. To capture a snapshot of the queried nucleotide sequencing taskset, the sequencing status system determines statuses of various nucleotide sequencing tasks, including a sequencing run, a data-analysis transfer of base-call data generated during the sequencing run, and a variant analysis of the base-call data. Based on the determined statuses, the sequencing status system provides a graphical status summary for the nucleotide sequencing taskset—including a run status icon indicating a status of the sequencing run, a data-transfer-status icon indicating a status of the data-analysis transfer, and a variant-analysis-status icon indicating a status of the variant analysis. By determining task statuses for various different nucleotide sequencing tasksets, the sequencing status system can generate different graphical status summaries for different nucleotide sequencing tasksets to display in a single, integrated graphical user interface—that is, an active sequencing interface.

As described further below, in some embodiments, the sequencing status system is executed by a local server corresponding to a sequencing device. As software runs on the local server, the sequencing status system can efficiently detect the status of sequencing runs from a nearby and connected sequencing device. As part of the local server, the sequencing status system can also quickly determine the status of (i) a data-analysis transfer of base-call data generated by a completed sequencing run and transferred to the local server and (ii) a variant analysis of the base-call data by complimentary software executed by the local server. Similarly, the sequencing status system can query the sequencing device (or other computing devices) to determine the status of external-data transfers of the base-call data or results data from the variant analysis to various external storages.

Regardless of whether the sequencing status system is run by a local server, a remote server, or other computing device, the sequencing status system can generate a group of dynamic status icons that together form a graphical status summary for a nucleotide sequencing taskset. In such a graphical status summary, for instance, the sequencing status system can change (or cause to change) the dynamic status icons that are ordered in positions representing different tasks from the nucleotide sequencing taskset. Such dynamic status icons can include, for instance, different icons in different ordered positions representing statuses for one or more of a sequencing run, an external-call-data transfer of base-call data generated during the sequencing run to an external storage, a data-analysis transfer of the base-call data to a computing device (e.g., local server) for a variant analysis, the variant analysis, or an external-analysis-data transfer of data generated during the variant analysis to an external storage.

In addition to grouping icons in ordered positions for various tasks, in some embodiments, the sequencing status system changes the icon representing an individual nucleotide sequencing task according to a detected status. Based on a change in detected status, for instance, the sequencing status system can cause the display of a different icon for the task in the corresponding position (as the status changes) to indicate the task having started, scheduled, progressed, completed, stopped, or an error. As the status of other nucleotide sequencing tasks change, the sequencing status system can likewise change to a different corresponding icon to represent the changed status of the task. At each position for a task in an ordered graphical status summary, therefore, the sequencing status system can indicate a not-started status, a scheduled status, or other status with a different and corresponding icon.

As further indicated above, the sequencing status system can present snapshot sequencing metrics along with a graphical status summary in an active sequencing interface. Such sequencing metrics may represent collective metrics giving a quality or statistical snapshot of a particular nucleotide sequencing taskset. For instance, as part of the active sequencing interface, the sequencing status system can identify and surface a collective base-call-quality metric indicating an accuracy of base calls generated during a sequencing run within a nucleotide sequencing taskset. Similarly, as part of the active sequencing interface, the sequencing status system can identify and surface a collective pass filter metric indicating a subset of base calls generated during the sequencing run that satisfy a quality filter.

In addition to snapshot sequencing metrics, in some cases, the sequencing status system provides options in an active sequencing interface to intervene in a nucleotide sequencing task for a given nucleotide sequencing taskset. For instance, the sequencing status system can intelligently surface a cancel option or a re-initiate option for a particular task or a particular nucleotide sequencing taskset. Based on the status indicated by a given dynamic status icon in a graphical status summary, therefore, the sequencing status system can provide intuitive selectable options for technicians to intervene (or otherwise manage) a nucleotide sequencing taskset.

As indicated above, the sequencing status system provides several technical benefits relative to existing sequencing management systems, such as by improving the efficiency and functionality of graphical user interfaces relative to existing sequencing management systems. For instance, in some embodiments, the sequencing status system generates and surfaces an efficient snapshot of a nucleotide sequencing taskset in the form of a graphical status summary. As indicated above, some existing sequencing management systems provide graphics that omit important stages of a sequencing process for genome samples, such as by including information about a sequencing run but omitting information about the data produced by the sequencing run or produced by a corresponding sequencing-data analysis for variants. In contrast to omitting such information, the sequencing status system queries and integrates status information for tasks in a nucleotide sequencing taskset into a visually efficient summary for the nucleotide sequencing taskset. In a series or string of dynamic status icons, for instance, the disclosed graphical status summary can quickly communicate the status of individual tasks from a nucleotide sequencing taskset, such as a sequencing run, a data transfer of base-call data, a variant analysis, and/or other nucleotide sequencing tasks. Similarly, the disclosed active sequencing interface can efficiently visualize collective sequencing metrics for a nucleotide sequencing taskset alongside a corresponding graphical status summary. By surfacing one or both of a graphical status summary and collective sequencing metrics, the sequencing status system (i) obviates the cumbersome graphical interface navigation that hinders existing sequencing management systems and (ii) efficiently communicates status information for tasks that such existing systems surface only through multiple clicks or navigation steps.

In addition or in the alternative to providing an overall snapshot of a sequencing process, in some embodiments, the sequencing status system generates and surfaces dynamic status icons for individual tasks from a nucleotide sequencing taskset that efficiently communicate the status of the corresponding task. As noted above, some existing sequencing management systems omit information concerning important stages of a sequencing process in graphic grids or other selective status graphics. For instance, existing graphic grids give no indication of processes in between a sequencing run and a sequencing-data analysis or processes after the sequencing-data analysis. By contrast, the sequencing status system generates and integrates dynamic status icons for important tasks from a nucleotide sequencing taskset that existing systems omit. For instance, in some cases, a graphical status summary includes a data-transfer-status icon indicating a status of a data-analysis transfer of base-call data from a sequencing device to a computing device for variant analysis. Because such a data-analysis transfer is often necessary for a sequencing process to progress, unlike existing systems, the sequencing status system efficiently represents the status of such a data-analysis transfer in a compact visual summary. Similarly, unlike existing systems, the sequencing status system can generate and surface separate icons in a graphical status summary for (i) an external-call-data transfer of base-call data generated during the sequencing run to an external storage or (ii) an external-analysis-data transfer of data generated during the variant analysis to an external storage. By surfacing dynamic status icons in a graphical status summary, the sequencing status system obviates cumbersome interface-upon-interface navigation that hinders existing sequencing management systems.

Beyond more efficient graphical status summaries, in certain cases, the sequencing status system intelligently surfaces options—unavailable at the summary level for existing sequencing management systems—in an active sequencing interface to intervene in a nucleotide sequencing task. As suggested above, existing systems often limit summary level options to cancel an ongoing sequencing run or another nucleotide sequencing task. By contrast, the sequencing status system intelligently surfaces selectable options based on (and relevant to) a particular graphical status summary. For instance, the sequencing status system can provide a selectable option to pause or re-initiate a particular nucleotide sequencing task in an active sequencing interface when a status of (and dynamic status icon indicates) the particular nucleotide sequencing task is experiencing an error or has stopped. Rather than a rigid set of generic cancel options in existing systems, therefore, the disclosed sequencing status system can intelligently surface selectable options for a particular nucleotide sequencing task based on a determined status for the particular task.

As illustrated by the foregoing discussion, the present disclosure utilizes a variety of terms to describe features and advantages of the sequencing status system. As used herein, for example, the term “nucleotide sequencing task” (or simply “task”) refers to an operation or a process performed by a computing device as part of determining a sequence of nucleotide bases for one or more sample genomes (or other nucleotide polymers) or part of saving data from determining such a sequence or from a corresponding analysis. In particular, a nucleotide sequencing task can include an operation or a process performed by a sequencing device that determines nucleotide-base sequences of fragments from a sample genome or performed by another computing device (e.g., server) to analyze data for the nucleotide-base sequences and determine variants within the nucleotide-base sequences with respect to a reference genome. A nucleotide sequencing task can likewise include an operation or a process of preserving data generated from determining a nucleotide-sequence (e.g., base-call data) or an analysis thereof. Accordingly, a nucleotide sequencing task can include, but is not limited to, a sequencing run, an external-call-data transfer of base-call data generated during the sequencing run to an external storage, a data-analysis transfer of the base-call data to a computing device (e.g., local server) for a variant analysis, the variant analysis, or an external-analysis-data transfer of data generated during the variant analysis to an external storage.

Relatedly, the term “nucleotide sequencing taskset” (or simply “taskset”) refers to a group of tasks performed by one or more computing devices that, as a collective process, determine a sequence of nucleotide bases for one or more sample genomes (or other nucleotide polymers) or save data from determining such a sequence or from a corresponding analysis. In particular, a nucleotide sequencing taskset can include a group of operations or processes (i) performed by a sequencing device to determine nucleotide-base sequences of fragments from a sample genome or save data related to the determined nucleotide-base sequences and (ii) performed by another computing device (e.g., server) to analyze data related to the determined nucleotide-base sequences, determine variants within the nucleotide-base sequences with respect to a reference genome, or save data resulting from the analyzed data. In some cases, a nucleotide sequencing taskset comprises tasks starting from a sequencing run that generates base-call data through completing (and storing a copy of) variant analysis of the base-call data.

As noted above, the sequencing status system can generate or provide a graphical status summary for a nucleotide sequencing taskset. As used in this disclosure, the term “graphical status summary” refers to one or more graphics (or data for such graphics) depicting a stage or progress of a nucleotide sequencing taskset. In particular, a graphical status summary includes a set of graphics that visually represent a current stage or progress of a set of nucleotide sequencing tasks that are part of a particular nucleotide sequencing taskset. In some cases, a graphical status summary includes individual status icons that depict a stage or progress of individual tasks from a nucleotide sequencing taskset. Accordingly, a graphical status summary can include a group of status icons that together depict a status or progress of a nucleotide sequencing taskset. Within proximity to or as part of a graphical status summary, in some embodiments, the sequencing status system provides a textual status summary for a particular nucleotide sequencing taskset (e.g., a textual-taskset-status summary) or a textual status summary for a particular nucleotide sequencing task (e.g., a textual-task-status summary).

Relatedly, a “status icon” refers to a graphical-user-interface element or graphic that depicts a stage or progress of a nucleotide sequencing task. For instance, a status icon can include a graphic, located at a designated position within a graphical status summary, that visually represents a determined status of a nucleotide sequencing task. As indicated above, a status icon can be dynamic and change to represent a changed status of a nucleotide sequencing task. For instance, the sequencing status system can change a dynamic status icon to represent that a nucleotide sequencing task has started, been scheduled, progressed, completed, stopped, or encountered an error. In some cases, a dynamic status icon may also be depicted in a particular color (e.g., green for completed, blue for in progress, grey for not started or scheduled, red for stopped or in error). Although not described in this paragraph, a status icon can also represent other suitable statuses.

As further used herein, the term “sequencing run” refers to an iterative process on a sequencing device to determine a primary structure of nucleotide fragments from a sample (e.g., genomic sample). In particular, a sequencing run includes cycles of sequencing chemistry and imaging performed by a sequencing device that incorporate nucleotide bases into growing oligonucleotides to determine nucleotide-fragment reads from nucleotide fragments extracted from a sample and seeded throughout a nucleotide-sample slide. In some cases, a sequencing run includes replicating nucleotide fragments from one or more genome samples seeded in clusters throughout a nucleotide-sample slide (e.g., a flow cell). Such a sequencing run can be a Sequencing By Synthesis (SBS) run. Further, upon completing a sequencing run, a sequencing device can generate base-call data in a file.

As just suggested, the term “base-call data” refers to data representing nucleotide-base calls for nucleotide-fragment reads and/or corresponding sequencing metrics. For instance, base-call data includes textual data representing nucleotide-base calls for nucleotide-fragment reads as text, (e.g., A, C, G, T) along with corresponding base-call-quality metrics, depth metrics, and/or other sequencing metrics. In some cases, base-call data is formatted in a text file, such as a binary base call (BCL) sequence file or as a fast-all quality (FASTQ) file.

As noted above, in some embodiments, base-call data includes sequencing metrics. As used herein, the term “sequencing metric” refers to a quantitative measurement or score indicating a degree to which an individual nucleotide-base call (or a sequence of nucleotide-base calls) aligns, compares, or quantifies with respect to a genomic coordinate or genomic region of a reference genome, with respect to nucleotide-base calls from nucleotide-fragment reads, or with respect to external genomic sequencing or genomic structure. For instance, a sequencing metric includes a quantitative measurement or score indicating a degree to which (i) individual nucleotide-base calls align, map, or cover a genomic coordinate or reference base of a reference genome; (ii) nucleotide-base calls compare to reference or alternative nucleotide-fragment reads in terms of mapping, mismatch, base-call quality, or other raw sequencing metrics; or (iii) genomic coordinates or regions corresponding to nucleotide-base calls demonstrate mappability, repetitive base-call content, DNA structure, or other generalized metrics.

As further used herein, the term “nucleotide-sample slide” refers to a plate or slide comprising oligonucleotides for sequencing nucleotide segments for sample genomes or other sample nucleic-acid polymers. In particular, a nucleotide-sample slide can refer to a slide containing fluidic channels through which reagents and buffers can travel as part of sequencing. For example, in one or more embodiments, a nucleotide-sample slide includes a flow cell (e.g., a patterned flow cell or non-patterned flow cell) comprising small fluidic channels and short oligonucleotides complementary to adaptor sequences. As indicated above, a nucleotide-sample slide can include wells (e.g., nanowells) comprising clusters of oligonucleotides.

As suggested above, a flow cell or other nucleotide-sample slide can (i) include a device having a lid extending over a reaction structure to form a flow channel therebetween that is in communication with a plurality of reaction sites of the reaction structure and (ii) include a detection device that is configured to detect designated reactions that occur at or proximate to the reaction sites. A flow cell or other nucleotide-sample slide may include a solid-state light detection or imaging device, such as a Charge-Coupled Device (CCD) or Complementary Metal-Oxide Semiconductor (CMOS) (light) detection device. As one specific example, a flow cell may be configured to fluidically and electrically couple to a cartridge (having an integrated pump), which may be configured to fluidically and/or electrically couple to a bioassay system. A cartridge and/or bioassay system may deliver a reaction solution to reaction sites of a flow cell according to a predetermined protocol (e.g., sequencing-by-synthesis), and perform a plurality of imaging events. For example, a cartridge and/or bioassay system may direct one or more reaction solutions through the flow channel of the flow cell, and thereby along the reaction sites. At least one of the reaction solutions may include four types of nucleotides having the same or different fluorescent labels. The nucleotides may bind to the reaction sites of the flow cell, such as to corresponding oligonucleotides at the reaction sites. The cartridge and/or bioassay system may then illuminate the reaction sites using an excitation light source (e.g., solid-state light sources, such as light-emitting diodes (LEDS)). The excitation light may provide emission signals (e.g., light of a wavelength or wavelengths that differ from the excitation light and, potentially, each other) that may be detected by the light sensors of the flow cell.

As further used herein, the term “nucleotide-fragment read” refers to an inferred sequence of one or more nucleotide bases (or nucleobase pairs) from all or part of a sample nucleotide sequence. In particular, a nucleotide-fragment read includes a determined or predicted sequence of nucleotide-base calls for a nucleotide fragment (or group of monoclonal nucleotide fragments) from a sequencing library corresponding to a genome sample. For example, in some cases, a sequencing device determines a nucleotide-fragment read by generating nucleotide-base calls for nucleotide bases passed through a nanopore of a nucleotide-sample slide, determined via fluorescent tagging, or determined from a cluster in a flow cell.

As further used herein, the term “variant analysis” refers to a secondary and/or a tertiary analysis of base-call data performed by a computing device to align nucleotide-fragment reads with a reference genome, determine genetic variants based on the aligned nucleotide-fragment reads, and/or interpret the determined genetic variants. For example, a variant analysis can include a secondary analysis performed by a server executing variant-call software to align samples' nucleotide-fragment reads with a reference genome, determine genetic variants of samples based on the aligned nucleotide-fragment reads with respect to the reference genome, and determine one or more of quality metrics, allele frequency metrics, or other sequencing metrics. As a further example, a variant analysis can include a tertiary analysis performed by a server executing bioinformatics software to determine potential genetic diseases (or genetic factors correlating with genetic diseases) based on determined genetic variants of a sample.

As indicated above, the term “reference genome” refers to a digital nucleic-acid sequence assembled as a representative example (or representative examples) of genes for an organism. Regardless of the sequence length, in some cases, a reference genome represents an example set of genes or a set of nucleic-acid sequences in a digital nucleic-acid sequenced determined by scientists or statistical models as representative of an organism of a particular species. For example, a linear human reference genome may be GRCh38 or other versions of reference genomes from the Genome Reference Consortium. As a further example, a reference genome may include a reference graph genome that includes both a linear reference genome and paths representing nucleic-acid sequences from ancestral haplotypes, such as Illumina DRAGEN Graph Reference Genome hg19.

As further used herein, the term “nucleotide-base call” (or simply “base call”) refers to a determination or prediction of a particular nucleotide base (or nucleotide-base pair) for a genomic coordinate of a sample genome or for an oligonucleotide during a sequencing cycle. In particular, a nucleotide-base call can indicate (i) a determination or prediction of the type of nucleotide base that has been incorporated within an oligonucleotide on a nucleotide-sample slide (e.g., read-based nucleotide-base calls) or (ii) a determination or prediction of the type of nucleotide base that is present at a genomic coordinate or region within a genome, including a variant call or a non-variant call in a digital output file. In some cases, for a nucleotide-fragment read, a nucleotide-base call includes a determination or a prediction of a nucleotide base based on intensity values resulting from fluorescent-tagged nucleotides added to an oligonucleotide of a nucleotide-sample slide (e.g., in a cluster of a flow cell). Alternatively, a nucleotide-base call includes a determination or a prediction of a nucleotide base from chromatogram peaks or electrical current changes resulting from nucleotides passing through a nanopore of a nucleotide-sample slide. By contrast, a nucleotide-base call can also include a final prediction of a nucleotide base at a genomic coordinate of a sample genome for a variant call file or other base-call-output file-based on nucleotide-fragment reads corresponding to the genomic coordinate. Accordingly, a nucleotide-base call can include a base call corresponding to a genomic coordinate and a reference genome, such as an indication of a variant or a non-variant at a particular location corresponding to the reference genome. Indeed, a nucleotide-base call can refer to a variant call, including but not limited to, a single nucleotide variant (SNV), an insertion or a deletion (indel), or base call that is part of a structural variant. As suggested above, a single nucleotide-base call can be an adenine (A) call, a cytosine (C) call, a guanine (G) call, or a thymine (T) call.

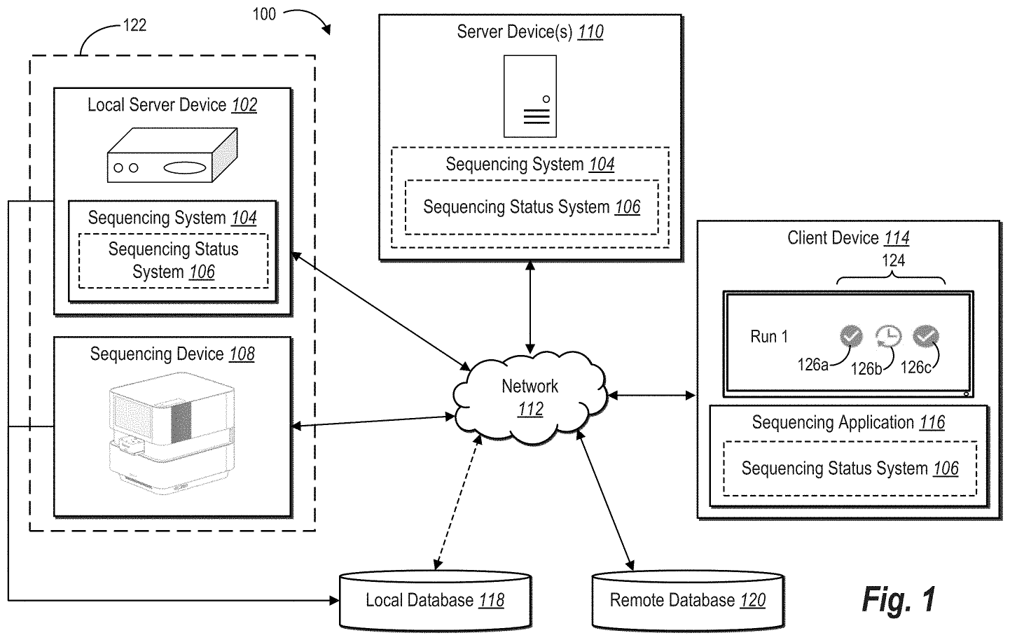

The following paragraphs describe the sequencing status system with respect to illustrative figures that portray example embodiments and implementations. For example, FIG. 1 illustrates a schematic diagram of a system environment (or “environment”) 100 in which a sequencing status system 106 operates in accordance with one or more embodiments. As illustrated, the environment 100 includes a local server device 102 connected to one or more server device(s) 110, a sequencing device 108, and a client device 114 via a network 112. While FIG. 1 shows an embodiment of the sequencing status system 106, this disclosure describes alternative embodiments and configurations below.

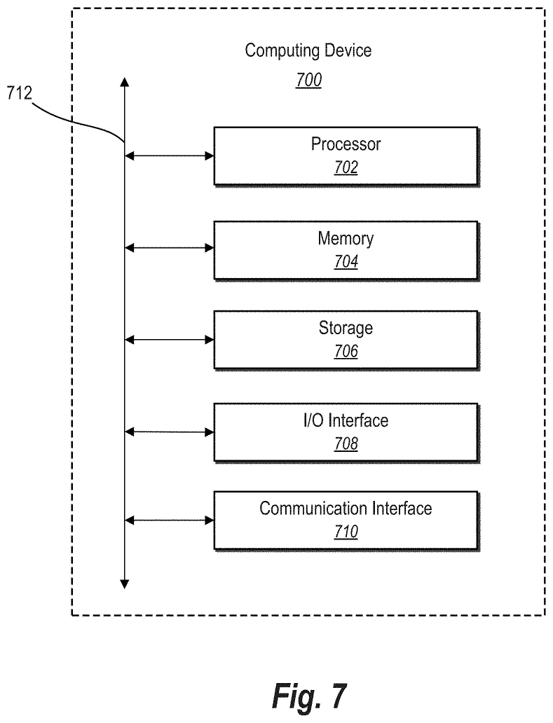

As shown in FIG. 1, the local server device 102, the sequencing device 108, the server device(s) 110, and the client device 114 can communicate with each other via the network 112. The network 112 comprises any suitable network over which computing devices can communicate. Example networks are discussed in additional detail below with respect to FIG. 7.

As indicated by FIG. 1, the sequencing device 108 comprises a device for sequencing a genomic sample or other nucleic-acid polymer. In some embodiments, the sequencing device 108 analyzes nucleic-acid segments or oligonucleotides extracted from genomic samples to generate nucleotide-fragment reads or other data utilizing computer implemented methods and systems (described herein) either directly or indirectly on the sequencing device 108. More particularly, the sequencing device 108 receives nucleotide-sample slides (e.g., flow cells) comprising nucleotide fragments extracted from samples and then copies and determines the nucleotide-base sequence of such extracted nucleotide fragments. In one or more embodiments, the sequencing device 108 utilizes SBS to sequence nucleic-acid polymers into nucleotide-fragment reads. In addition or in the alternative to communicating across the network 112, in some embodiments, the sequencing device 108 bypasses the network 112 and communicates directly with the local server device 102 or the client device 114.

As further indicated by FIG. 1, the local server device 102 is located at or near a same physical location of the sequencing device 108. Indeed, in some embodiments, the local server device 102 and the sequencing device 108 are integrated into a same computing device, as indicated by dotted lines 122. The local server device 102 may run a sequencing system 104 to generate, receive, analyze, store, and transmit digital data, such as by receiving base-call data or determining variant calls based on analyzing such base-call data. As shown in FIG. 1, the sequencing device 108 may send (and the local server device 102 may receive) base-call data generated during a sequencing run of the sequencing device 108. By executing software from the form of the sequencing system 104, the local server device 102 may align nucleotide-fragment reads with a reference genome and determine genetic variants based on the aligned nucleotide-fragment reads. The local server device 102 may also communicate with the client device 114. In particular, the local server device 102 can send data to the client device 114, including status information for nucleotide sequencing tasksets, a variant call file (VCF), or other information indicating nucleotide-base calls, sequencing metrics, error data, or other metrics.

As further indicated by FIG. 1, the server device(s) 110 are located remotely from the local server device 102 and the sequencing device 108. Similar to the local server device 102, in some embodiments, the server device(s) 110 include a version of the sequencing system 104. Accordingly, the server device(s) 110 may generate, receive, analyze, store, and transmit digital data, such as data for determining nucleotide-base calls or sequencing nucleic-acid polymers. Similarly, the sequencing device 108 may send (and the server device(s) 110 may receive) base-call data from the sequencing device 108. The server device(s) 110 may also communicate with the client device 114. In particular, the server device(s) 110 can send data to the client device 114, including status information for nucleotide sequencing tasksets, VCFs, or other sequencing related information.

In some embodiments, the server device(s) 110 comprise a distributed collection of servers where the server device(s) 110 include a number of server devices distributed across the network 112 and located in the same or different physical locations. Further, the server device(s) 110 can comprise a content server, an application server, a communication server, a web-hosting server, or another type of server.

As further illustrated and indicated in FIG. 1, the client device 114 can generate, store, receive, and send digital data. In particular, the client device 114 can receive status data from the local server device 102 or receive sequencing metrics from the sequencing device 108. Furthermore, the client device 114 may communicate with the local server device 102 or the server device(s) 110 to receive a VCF comprising nucleotide base calls and/or other metrics, such as a base-call-quality metrics or pass-filter metrics. The client device 114 can accordingly present or display information pertaining to variant calls or other nucleotide-base calls within a graphical user interface to a user associated with the client device 114. For example, the client device 114 can present an active sequencing interface comprising a graphical status summary of a nucleotide sequencing taskset.

Although FIG. 1 depicts the client device 114 as a desktop or laptop computer, the client device 114 may comprise various types of client devices. For example, in some embodiments, the client device 114 includes non-mobile devices, such as desktop computers or servers, or other types of client devices. In yet other embodiments, the client device 114 includes mobile devices, such as laptops, tablets, mobile telephones, or smartphones. Additional details regarding the client device 114 are discussed below with respect to FIG. 7.

As further illustrated in FIG. 1, the client device 114 includes a sequencing application 116. The sequencing application 116 may be a web application or a native application stored and executed on the client device 114 (e.g., a mobile application, desktop application). The sequencing application 116 can include instructions that (when executed) cause the client device 114 to receive data from the sequencing status system 106 and present, for display at the client device 114, data concerning a status of a nucleotide sequencing taskset or data from a VCF. Furthermore, the sequencing application 116 can instruct the client device 114 to display graphical status summaries for multiple nucleotide sequencing tasksets.

As further illustrated in FIG. 1, a version of the sequencing status system 106 may be located on the client device 114 as part of the sequencing application 116 or on the server device(s) 110. Accordingly, in some embodiments, the sequencing status system 106 is implemented by (e.g., located entirely or in part) on the client device 114. In yet other embodiments, the sequencing status system 106 is implemented by one or more other components of the environment 100, such as the server device(s) 110. In particular, the sequencing status system 106 can be implemented in a variety of different ways across local server device 102, the sequencing device 108, the client device 114, and the server device(s) 110. For example, the sequencing status system 106 can be downloaded from the server device(s) 110 to the local server device 102 and/or the client device 114 where all or part of the functionality of the sequencing status system 106 is performed at each respective device within the environment 100.

As further illustrated in FIG. 1, the environment 100 includes a local database 118 and a remote database 120. As the names suggest, the local database 118 is located at or nearby a same location as the sequencing device 108, whereas the remote database 120 is location at a different and remote location. The local database 118 and the remote database 120 can store information, such as base-call data, variant call files, data for sample nucleotide sequences, and sequencing metrics. In some embodiments, the local server device 102, the sequencing device 108, the server device(s) 110, and/or the client device 114 communicate with the local database 118 and the remote database 120 (e.g., via the network 112) to store and/or access information, such base-call data, variant call files, data for sample nucleotide sequences, and sequencing metrics.

As further shown in FIG. 1, in one or more embodiments, the sequencing status system 106 generates a graphical status summary 124 of a nucleotide sequencing taskset for display on the client device 114. As executed by the local server device 102 or the server device(s) 110, for instance, the sequencing status system 106 receives a status query from the client device 114 concerning a nucleotide sequencing taskset. To capture a snapshot of the queried nucleotide sequencing taskset, the sequencing status system 106 determines statuses of various nucleotide sequencing tasks within the nucleotide sequencing taskset, including a status of a sequencing run from the sequencing device 108, a data-analysis transfer of base-call data from the sequencing device 108 to the local server device 102 or to the server device(s) 110, and a variant analysis of the base-call data by the local server device 102 or by the server device(s) 110.

Based on the determined statuses, the sequencing status system 106 provides the graphical status summary 124 for the nucleotide sequencing taskset—including data for a run status icon 126a indicating a status of the sequencing run, a data-transfer-status icon 126b indicating a status of the data-analysis transfer, and a variant-analysis-status icon 126c indicating a status of the variant analysis. As described below, such graphical status summaries can be displayed on other computing devices, such as the sequencing device 108, a local computing device connected to the local server device 102, or an external computing device connected to the server device(s) 110 or the local server device 102 through the network 112. Graphical status summaries can take various different forms. While the graphical status summary 124, for instance, includes status icons at designated positions for three different nucleotide sequencing tasks, other graphical status summaries may include status icons for four, five, or another number of nucleotide sequencing tasks.

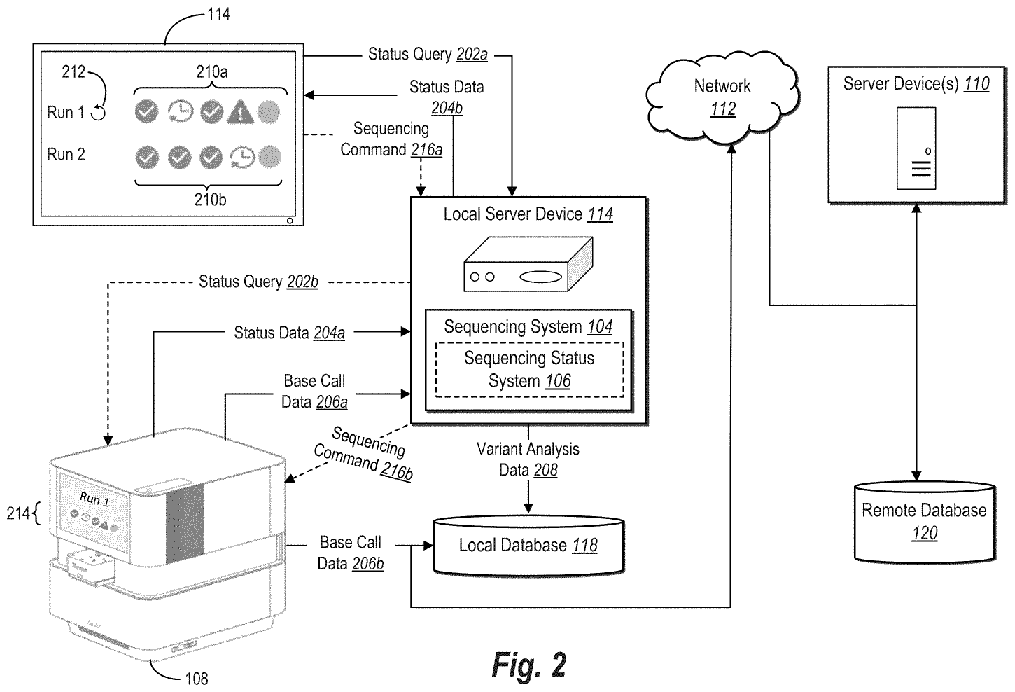

As indicated above, the sequencing status system 106 can exchange and orchestrate data across multiple computing devices to determine the status of nucleotide sequencing tasksets and generate graphical status summaries for such tasksets. In accordance with one or more embodiments, FIG. 2 illustrates an example of the sequencing status system 106 (i) querying other computing devices for the status of various nucleotide sequencing tasks that are part of a nucleotide sequencing taskset and (ii) generating a graphical status summary for the nucleotide sequencing taskset.

As shown in FIG. 2, for instance, the client device 114 sends a status query 202a to the local server device 102 inquiring of a status of a nucleotide sequencing taskset. In some embodiments, the status query 202a includes data identifiers for each nucleotide sequencing task in a nucleotide sequencing taskset to prompt the sequencing status system 106 to determine a status for each nucleotide sequencing task. By contrast, in certain embodiments, the status query 202a includes a data identifier for the particular nucleotide sequencing taskset that prompts the sequencing status system 106 to determine a status for each nucleotide sequencing task.

As further shown in FIG. 2, the sequencing status system 106 determines a status of tasks that are part of the nucleotide sequencing taskset-based on receiving the status query 202a. For instance, as shown in FIG. 2, the sequencing status system 106 sends a status query 202b to the sequencing device 108 inquiring of statuses for one or more of a sequencing run, a data-analysis transfer of base-call data, or an external-call-data transfer of the base-call data to the local database 118 or other external storage. Based on receiving the status query 202b from the local server device 102, the sequencing device 108 determines whether the sequencing run has started, been scheduled, progressed, completed, stopped, or encountered an error and, subsequently, sends status data 204a to the local server device 102 indicating a corresponding status. Similarly, also based on receiving the status query 202b, in some embodiments, the sequencing device 108 determines whether it has started, scheduled, progressed, completed, stopped, or encountered an error in sending base-call data 206a generated during the sequencing run to the local server device 102 and/or sending base-call data 206b to the local database 118.

In addition to communicating with the sequencing device 108 to determine a status of nucleotide sequencing tasks, as suggested by FIG. 2, the sequencing status system 106 also detects a status of tasks that are part of the nucleotide sequencing taskset from either the local server device 102 or the server device(s) 110. For instance, the sequencing status system 106 imports or determines, from one or more storage devices on the local server device 102, statuses for one or more of a data-analysis transfer of base-call data to the local server device 102, a variant analysis of the base-call data, or an external-analysis-data transfer of variant analysis data 208 generated during the variant analysis to the local database 118 or the remote database 120 or other external storage. In the alternative to an internal query on the local server device 102, in some cases, the sequencing status system 106 sends a status query to the server device(s) 110 to determine a status for the data-analysis transfer, the variant analysis, and/or the external-analysis-data transfer. Based on receiving the status query 202a from the client device 114, the sequencing status system 106 determines whether the data-analysis transfer, the variant analysis, and/or the external-analysis-data transfer has started, been scheduled, progressed, completed, stopped, or encountered an error.

As further indicated by FIG. 2, after determining statuses of the tasks in the nucleotide sequencing taskset, the sequencing status system 106 sends status data 204b to the client device 114—based on (or in response to) the status query 202a. In some embodiments, the status data 204b includes data indicators for a status of each nucleotide sequencing task within a taskset. By contrast, in certain cases, the status data 204b includes data indicators for an updated or changed status of one or more nucleotide sequencing tasks, but no data indicators for nucleotide sequencing tasks with an unchanged status from a previous status query.

As indicated above, the status data 204b from the local server device 102 can prompt the client device 114 to present a graphical status summary 210a. Based on receiving the status data 204b from the local server device 102, in some cases, the client device 114 presents the graphical status summary 210a for the queried nucleotide sequencing taskset. As shown in FIG. 2, the graphical status summary 210a includes a group of status icons that together indicate a status of the queried nucleotide sequencing taskset. For instance, the graphical status summary 210a includes a dynamic status icon for each of a sequencing run, an external-call-data transfer of base-call data to the local database 118 or other external storage, a data-analysis transfer of the base-call data to the local server device 102 for a variant analysis, the variant analysis, and an external-analysis-data transfer of variant analysis data 208 generated during the variant analysis to the local database 118 or the remote database 120 or other external storage. As the client device 114 sends additional status queries as a data pull or receives additional status queries as a data push, the client device 114 can change the dynamic status icon for one or more tasks within the queried nucleotide sequencing taskset.

As suggested above, the sequencing status system 106 can facilitate status queries from different computing devices and provide a graphical status summary for display on different querying computing devices. As indicated by FIG. 2, for instance, the sequencing device 108 sends a status query for a nucleotide sequencing taskset and receives status data in response to the status query. Based on the status data, the sequencing device 108 likewise displays a graphical status summary 214 within a graphical user interface. As shown, the graphical status summary 214 matches the graphical status summary 210a for the same nucleotide sequencing taskset.

As further indicated by FIG. 2, the sequencing status system 106 can provide (and the client device 114 display) graphical status summaries for multiple queried nucleotide sequencing tasksets. Indeed, the client device 114 display a graphical status summary 210b for an additional queried nucleotide sequencing taskset. To determine a status of an additional nucleotide sequencing taskset, in some embodiments, the sequencing status system 106 sends or receives the same type of status queries or status data as described above for the graphical status summary 210a. Indeed, in some embodiments, the sequencing status system 106 can orchestrate in parallel the exchange of status queries and status data for different nucleotide sequencing tasksets to facilitate display of multiple corresponding graphical status summaries on a single computing device.

As suggested above, in some embodiments, the sequencing status system 106 provides selectable options, for display in the client device 114, to intervene in a task for a given nucleotide sequencing taskset. As shown in FIG. 2, for instance, the sequencing status system 106 provides a selectable option 212 to intervene in a targeted nucleotide sequencing task corresponding to a particular status icon in the graphical status summary 210a. To intelligently surface the selectable option 212, in certain implementations, the sequencing status system 106 identifies a status of a nucleotide sequencing task and identifies an operation corresponding to the status of the nucleotide sequencing task, such as by identifying a sequencing operation to re-initiate a nucleotide sequencing task based on a status indicating the task has stopped or encountered an error.

As shown in FIG. 2, for instance, the sequencing status system 106 sends data (e.g., as part of the status data 204b) identifying or triggering an option for a re-initiate operation to the client device 114 based on determining that a variant analysis has encountered an error. Based on receiving the data identifying or triggering the option for the re-initiate operation, the client device 114 displays a re-initiate option as the selectable option 212 for display alongside, nearby, or within a same graphical user interface as the graphical status summary 210a.

By intelligently providing selectable options to intervene in (or otherwise manage) different nucleotide sequencing tasks during the course of a taskset, the sequencing status system 106 gives tools to a computing device to manage a nucleotide sequencing taskset running across multiple devices. As further shown in FIG. 2, in certain implementations, the client device 114 sends a sequencing command 216a to the local server device 102 based on detecting a selection of the selectable option 212 to perform a sequencing operation for the targeted nucleotide sequencing task. For instance, the client device 114 sends the sequencing command 216a to the local server device 102 to re-initiate the variant analysis represented by a status icon in the graphical status summary 210a. In some embodiments, based on receiving the sequencing command 216a, the local server device 102 re-initiates the variant analysis of base-call data from the corresponding sequencing run.

As suggested above and explained further below, the sequencing status system 106 can intelligently determine and send data for display of options to intervene in a variety of nucleotide sequencing task for a given nucleotide sequencing taskset. For instance, in some cases, the client device 114 prompts the local server device 102 to send (or the client device 114 sends across the network 112) a sequencing command 216b to the sequencing device 108. As further shown in FIG. 2, based on receiving a sequencing command from the client device 114, the local server device 102 optionally sends the sequencing command 216b to the sequencing device 108 to perform a sequencing operation for a targeted nucleotide sequencing task, such as by re-initiating a sequencing run.

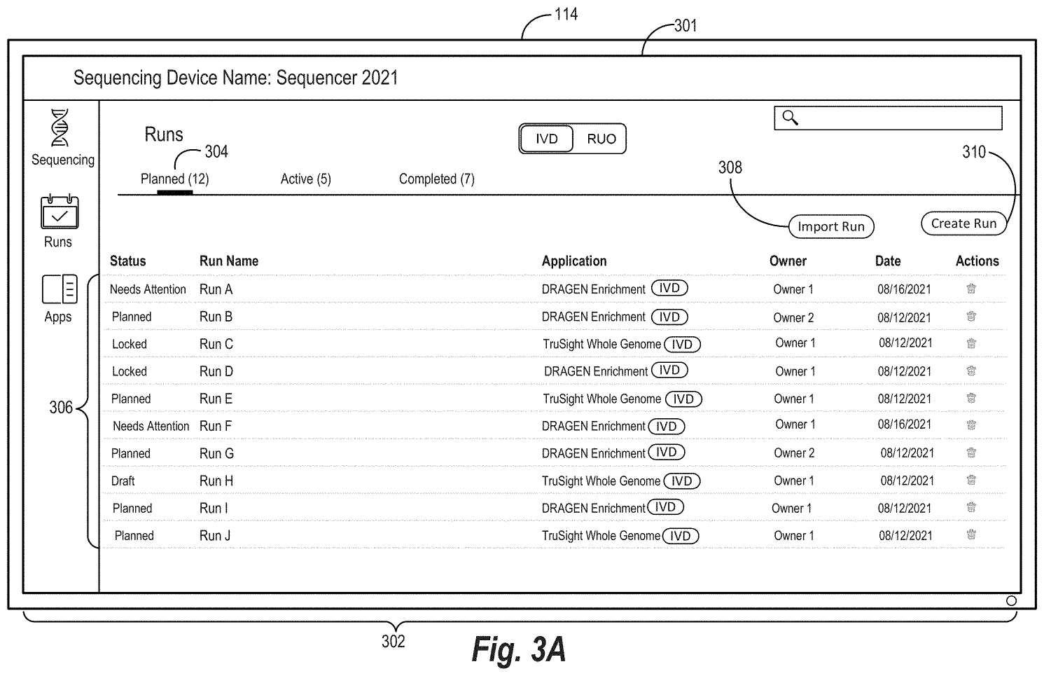

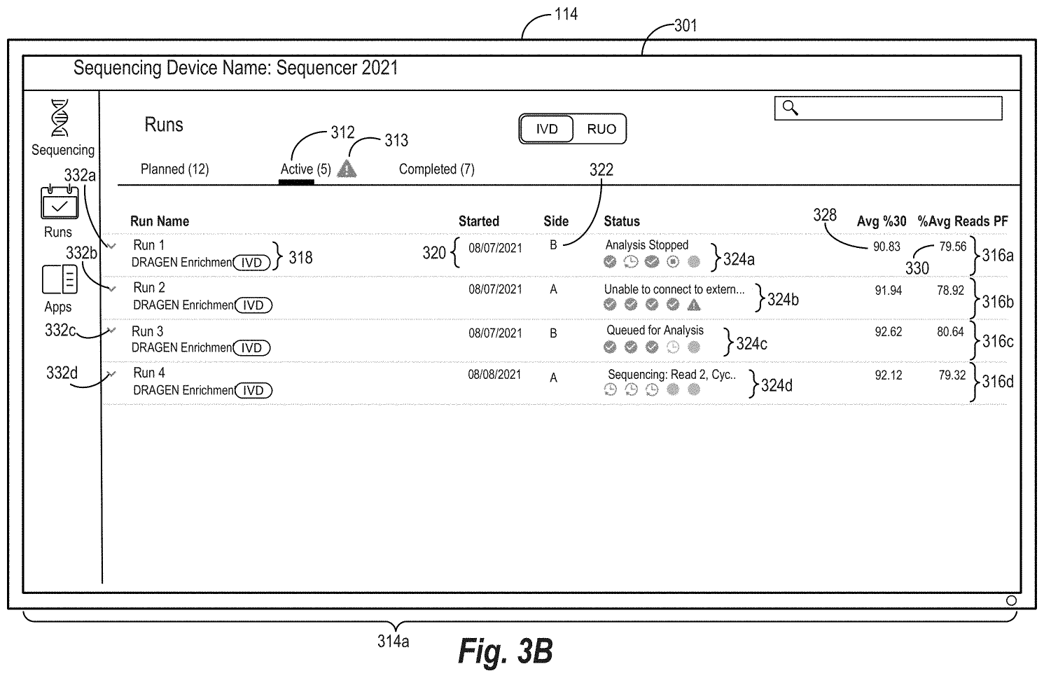

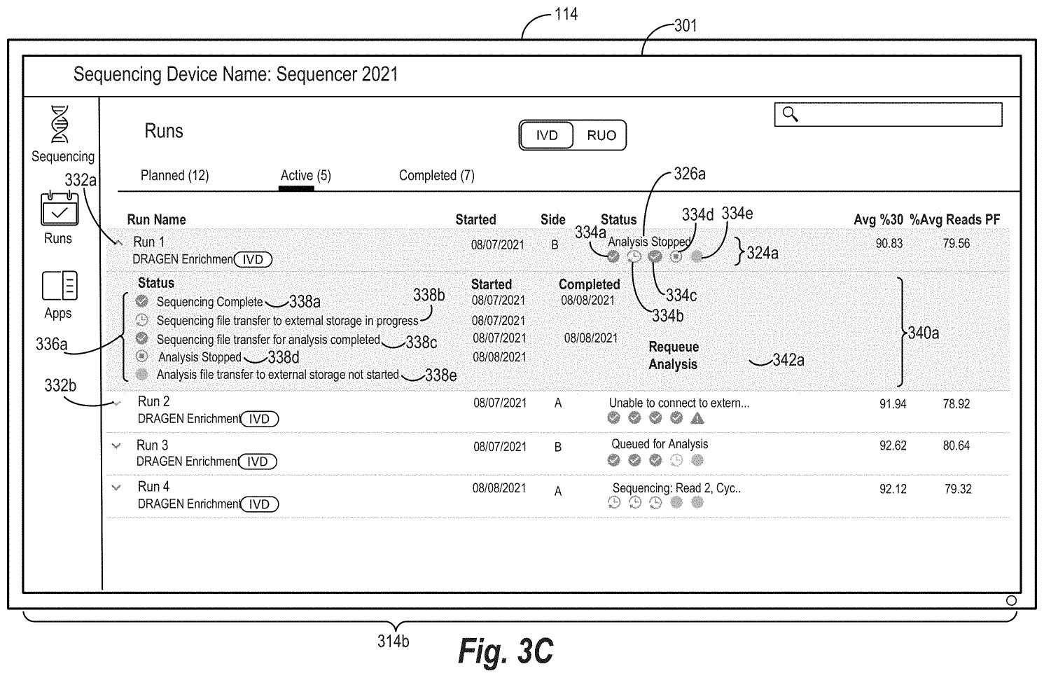

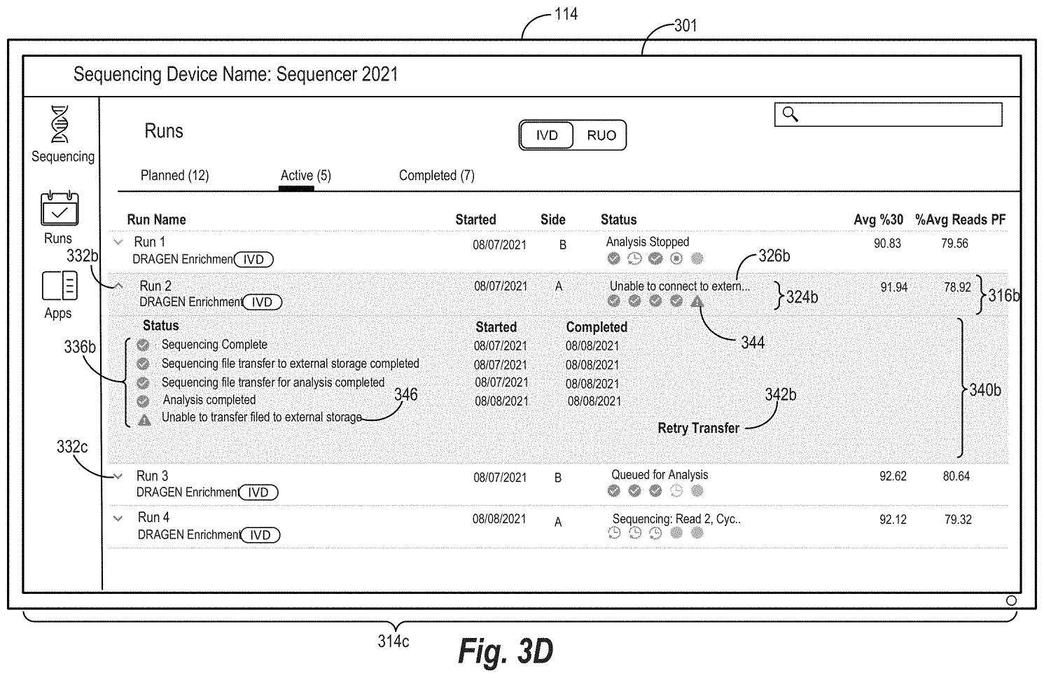

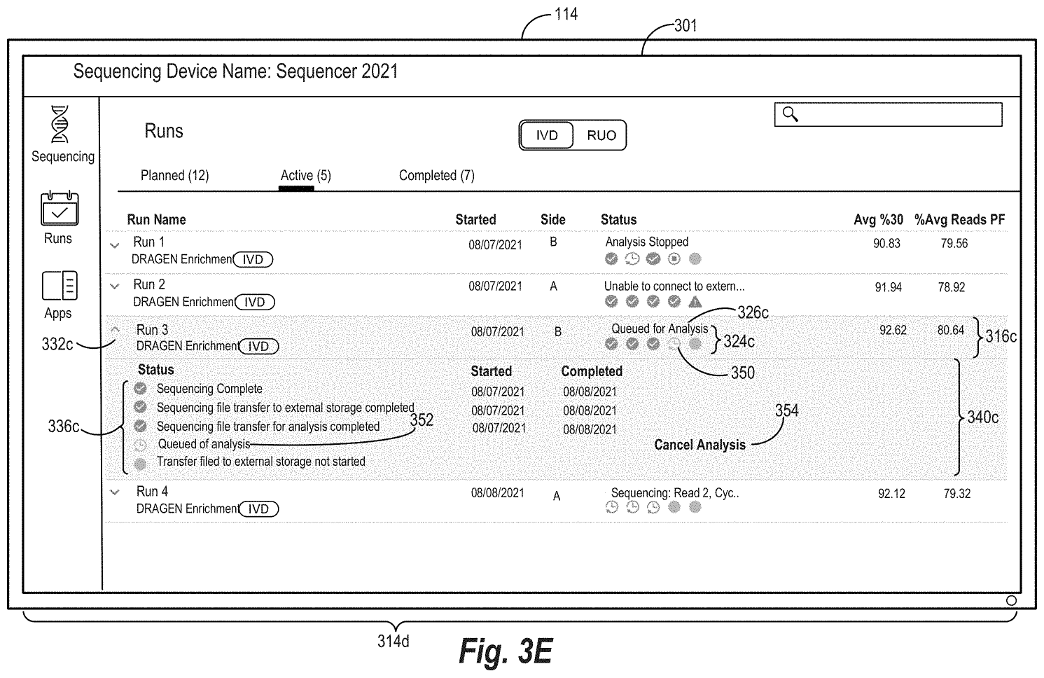

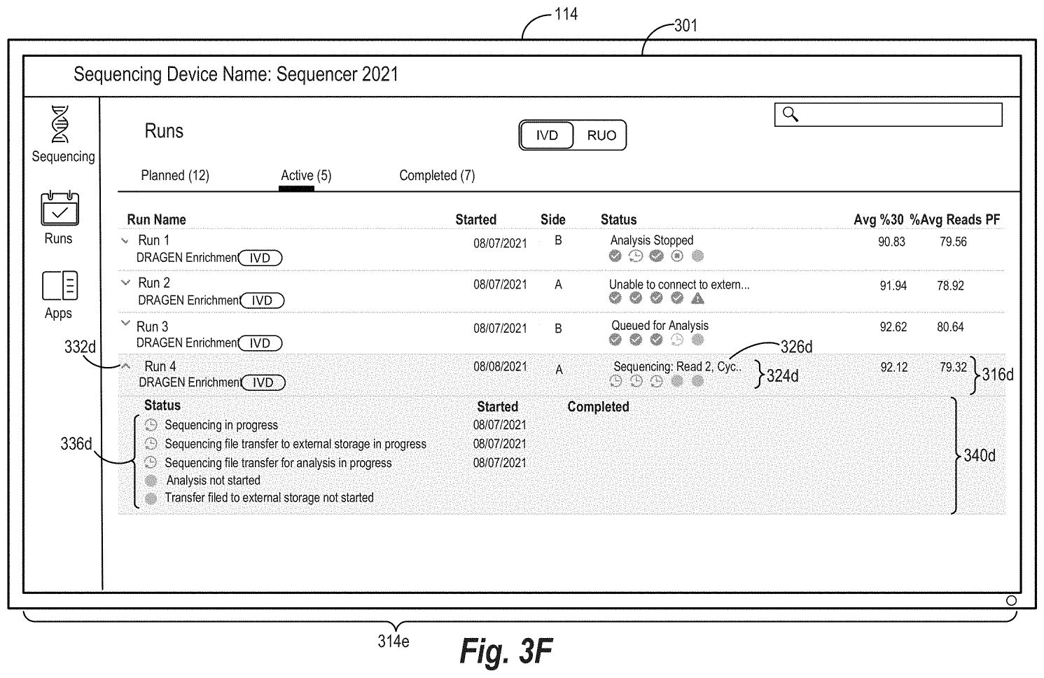

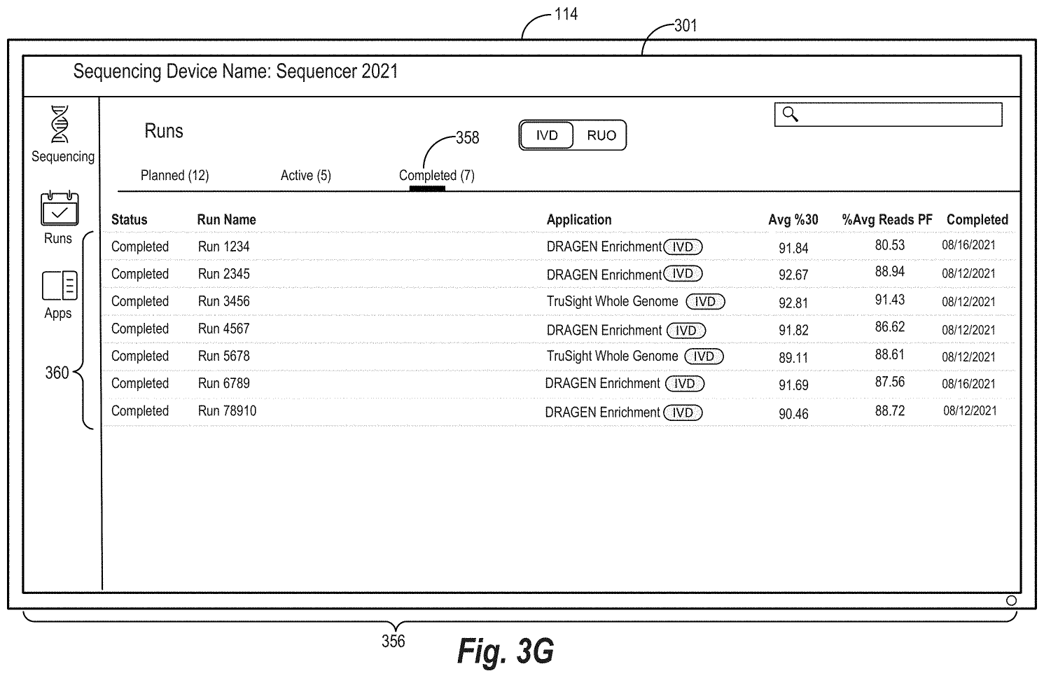

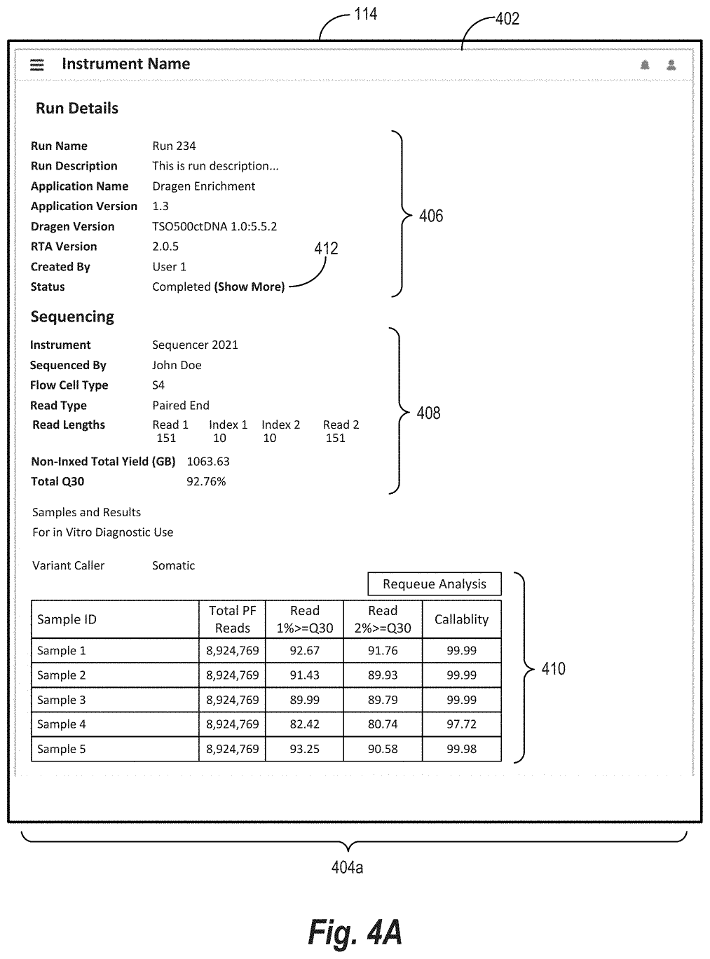

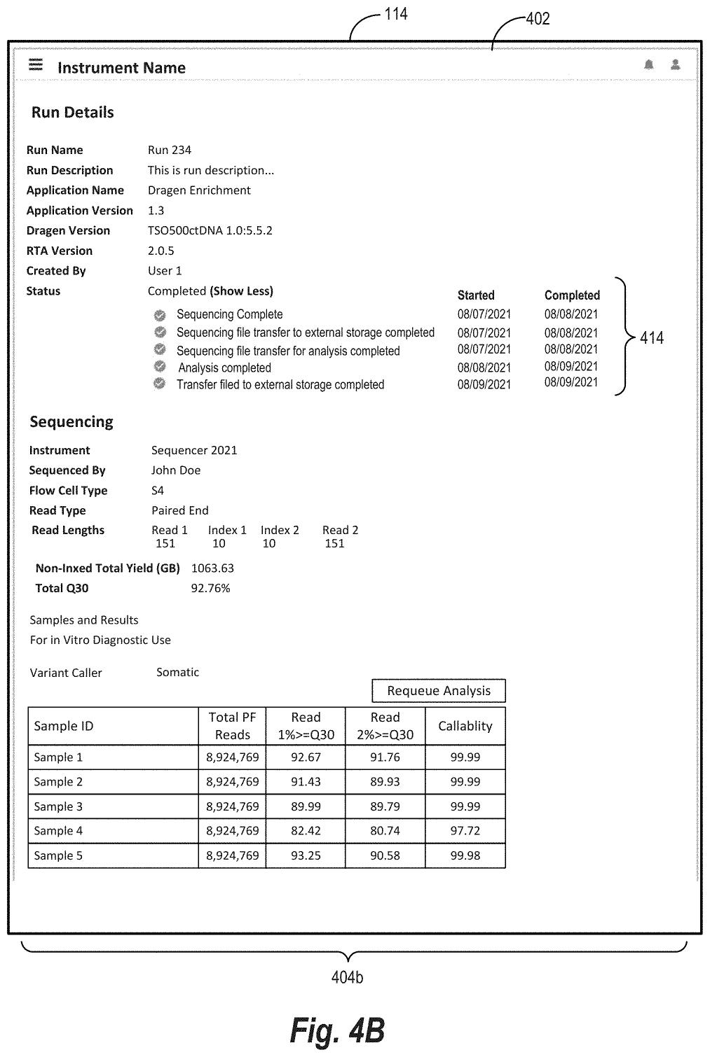

As suggested above, in certain embodiments, the sequencing status system 106 can orchestrate and manage different nucleotide sequencing tasksets at various stages. As shown in FIGS. 3A-3G and described below, the sequencing status system 106 can provide tools to plan, manage, intervene, and view summaries of different nucleotide sequencing tasksets starting from a sequencing run that generates base-call data through completing (and storing a copy of) variant analysis of the base-call data. FIGS. 3A-3G illustrate the client device 114 presenting graphical user interfaces for managing planned tasksets, active tasksets, and completed tasksets from the perspective of the client device 114 implementing computer-executable instructions of the sequencing application 116. FIGS. 4A-4B illustrate the client device 114 presenting graphical user interfaces comprising details for a nucleotide sequencing taskset. Rather than repeatedly refer to the computer-executable instructions causing a computing device to perform certain actions for the sequencing status system 106, this disclosure describes the client device 114 or the sequencing status system 106 performing those actions in the following paragraphs.

As shown in FIG. 3A, for instance, the client device 114 presents a planned sequencing interface 302 of the sequencing application 116 within a screen 301. Based on detecting a selection of a planned sequencing tab 304, for example, the client device 114 presents the planned sequencing interface 302. The planned sequencing interface 302 includes planned taskset overviews 306 for nucleotide sequencing tasksets that have been created or scheduled to begin at a future time. The planned taskset overviews 306 each in turn include high-level information concerning a planned nucleotide sequencing taskset, including a textual status summary (e.g., Planned, Locked), sequencing run name or nucleotide-sequencing-taskset name (e.g., Run A), secondary-analysis application type (e.g., DRAGEN Enrichment), owner, and date last modified.

As further shown in FIG. 3A, the planned sequencing interface 302 includes an import taskset option 308 and a create taskset option 310. Based on detecting a selection of the import taskset option 308, the client device 114 presents one or more graphical user interfaces to import a previously planned nucleotide sequencing taskset. By contrast, based on detecting a selection of the create taskset option 310, the client device 114 presents one or more graphical user interfaces to create a new nucleotide sequencing taskset.

In addition to presenting a snapshot of planned nucleotide sequencing tasksets, the sequencing status system 106 also presents a snapshot of active nucleotide sequencing tasksets in which at least a sequencing run has commenced. As shown in FIG. 3B, for instance, the client device 114 presents an active sequencing interface 314a of the sequencing application 116 within the screen 301. Based on detecting a selection of an active sequencing tab 312, for example, the client device 114 presents the active sequencing interface 314a. Indeed, in some cases, the client device 114 presents an error icon 313 alongside (or within proximity of) the active sequencing tab 312 to indicate an error or other issue with one or more active nucleotide sequencing tasksets can be viewed within the active sequencing interface 314a.

As further shown in FIG. 3B, the active sequencing interface 314a includes active taskset overviews 316a, 316b, 316c, and 316d. Each of the active taskset overviews 316a-316d include basic identifying information for a nucleotide sequencing taskset. For instance, each of the active taskset overviews 316a-316d include a run name or nucleotide-sequencing-taskset name, a start date, a nucleotide-sample-slide-side indicator, a graphical status summary, a collective base-call-quality metric, and a collective pass filter metric for a corresponding nucleotide sequencing taskset. Indeed, the active taskset overviews 316a, 316b, 316c, and 316d respectively include graphical status summaries 324a, 324b, 324c, and 324d. This disclosure describes each active taskset overview and graphical status summary in turn below.

As part of the active taskset overview 316a, for example, the client device 114 presents a nucleotide-sequencing-taskset name 318, a start date 320, and a nucleotide-sample-slide-side indicator 322. The nucleotide-sequencing-taskset name 318 includes a code, alphanumeric information, or other identifier for the nucleotide sequencing taskset. In some cases, the nucleotide-sequencing-taskset name 318 also includes an indicator for a type of nucleotide sequencing taskset, such as in vitro diagnostic (IVD) or research use only (RUO). The start date 320 includes a data that a sequencing device (or another computing device) commenced a nucleotide sequencing taskset. In some cases, the start date 320 indicates a date and time at which a sequencing device began a sequencing run. The nucleotide-sample-slide-side indicator 322 indicates a side of a nucleotide-sample slide comprising one or more samples corresponding to the relevant nucleotide sequencing taskset. The active taskset overview 316a indicates side A or side B of a nucleotide-sample slide because, in some cases, a particular sequencing run for a nucleotide sequencing taskset may correspond to a single slide.

In addition to the nucleotide-sequencing-taskset name 318 and other details, the client device 114 presents a graphical status summary 324a as part of the active taskset overview 316a. As shown in FIG. 3A, the graphical status summary 324a includes a set of status icons that together depict a stage or progress of an active nucleotide sequencing taskset. Indeed, the graphical status summary 324a orders the set of status icons in designated positions for different tasks within the active nucleotide sequencing taskset. By providing a specific status icon for each task and ordering the status icons in designated positions, the sequencing status system 106 provides a graphical snapshot that efficiently communicates both an overall status of the active nucleotide sequencing taskset and a status of individual tasks. This disclosure describes the specific status icons and designated positions further below with respect to FIG. 3B.

In addition to (or as part of) the graphical status summary 324a, the client device 114 intelligently presents a textual-taskset-status summary 326a within the active taskset overview 316a. As described above with respect to FIG. 2, the sequencing status system 106 can determine a status of each nucleotide sequencing task within a nucleotide sequencing task. Based on the status of each nucleotide sequencing task—or an overall status of the nucleotide sequencing taskset—the sequencing status system 106 can determine and provide the textual-taskset-status summary 326a for display within the active sequencing interface 314a. The textual-taskset-status summary 326a includes a textual description of the overall status of the active nucleotide sequencing taskset that compliments the graphical status summary 324a. For example, in some embodiments, the textual-taskset-status summary 326a describes a current status of a last scheduled or a last commenced nucleotide sequencing task according to an order of the nucleotide sequencing taskset. As shown in FIG. 3B, in some embodiments, the graphical status summary 324a depicts status icons in designated positions according to the order of the nucleotide sequencing taskset.

In addition to the graphical status summary 324a and the textual-taskset-status summary 326a, the sequencing status system 106 can provide (and the client device 114 present) collective sequencing metrics for the nucleotide sequencing taskset as part of the active taskset overview 316a. As shown in FIG. 3B, for instance, the client device 114 presents a collective base-call-quality metric 328 for the nucleotide sequencing taskset. To surface this collective sequencing metric, in some cases, the sequencing status system 106 or the sequencing system 104 determines a percentage of base calls for nucleotide-fragment reads (generated during the sequencing run) that satisfy (e.g., equal or exceed) a threshold base-call-quality metric. Such a threshold base-call-quality metric may be a quality metric of 30 according to a Phred algorithm (e.g., Phred quality score of Q30). In the alternative, the sequencing status system 106 or the sequencing system 104 determines a percentage of variant calls in a variant call file that satisfy a threshold base-call-quality metric. Regardless of the format or method, the collective base-call-quality metric 328 indicates an accuracy of base calls generated during a sequencing run (or an accuracy of variant calls in a VCF) that is part of the nucleotide sequencing taskset. Based on determining or receiving the collective base-call-quality metric 328, the sequencing status system 106 provides the collective base-call-quality metric 328 for display proximate to the graphical status summary 324a within the active sequencing interface 314a.

As further shown in FIG. 3B, the client device 114 presents a collective pass filter metric 330 for the nucleotide sequencing taskset. To surface this collective sequencing metric, in some cases, the sequencing status system 106 or the sequencing system 104 determines a percentage of nucleotide-fragment reads that pass a quality filter. For instance, the quality filter may be a chastity metric for nucleotide fragment reads. In some embodiments, a nucleotide-fragment read for a given cluster of oligonucleotides passes a chastity filter when no more than one base call has a chastity value below 0.6 in the initial twenty-five cycles of a sequencing run. Regardless of the format or method for passing filter, the collective pass filter metric 330 indicates a subset of base calls generated during the sequencing run that satisfy a quality filter. Based on determining or receiving the collective pass filter metric 330, the sequencing status system 106 provides the collective pass filter metric 330 for display proximate to the graphical status summary 324a within the active sequencing interface 314a.

As shown in FIG. 3B, each of the active taskset overviews 316b-316d include a same type of information as just described for the active taskset overview 316a. Accordingly, each of the active taskset overviews 316b-316d include a sequencing run name or nucleotide-sequencing-taskset name, start date, nucleotide-sample-slide-side indicator, a graphical status summary, and collective sequencing metrics—but for different active nucleotide sequencing tasksets. This disclosure describes certain differences among the active taskset overviews 316a-316d and graphical status summaries 324a-324d below.

In addition to a graphical status summary, in certain implementations, the sequencing status system 106 can provide an expanded graphical status summary comprising additional detail concerning an active nucleotide sequencing taskset. As shown by a transition from FIG. 3B to FIG. 3C, when the client device 114 detects a selection of an expand option 332a, the client device 114 presents an expanded active taskset overview 340a within an active sequencing interface 314b. As shown in FIG. 3C, the expanded active taskset overview 340a includes an expanded graphical status summary 336a with a textual-task-status summary for each dynamic status icon.

As shown in both FIGS. 3B and 3C, the graphical status summary 324a includes individual status icons arranged in a particular order with designated positions. As shown in FIG. 3C, for instance, the graphical status summary 324a includes a run status icon 334a located at a sequencing run position (or first designated position), an external-transfer-status icon 334b located at an external-call-data-transfer position (or second designated position), a data-transfer-status icon 334c located at data-analysis-transfer position (or third designated position), a variant-analysis-status icon 334d located at variant-analysis position (or fourth designated position), and an external-transfer-status icon 334e located at an external-analysis-data-transfer position (or fifth designated position).

As indicated above, an individual status icon at each designated position in the graphical status summary 324a can take the form different graphics to indicate different statuses of a nucleotide sequencing task. At the first designated position shown in the graphical status summary 324a, for instance, the run status icon 334a indicates a status of the sequencing run. In this example, the run status icon 334a is a circular check-mark icon representing a completed status. At the second designated position shown in the graphical status summary 324a, the external-transfer-status icon 334b indicates a status of an external-call-data transfer of the base-call data (generated during the sequencing run) to an external storage. In this example, the external-transfer-status icon 334b is a blue clock icon or a darker shaded clock icon representing an in-progress status. At the third designated position shown in the graphical status summary 324a, the data-transfer-status icon 334c indicates a status of a data-analysis transfer of base-call data (generated during the sequencing run) to a server for variant analysis. In this example, the data-transfer-status icon 334c is a circular check-mark icon representing a completed status.

As further shown in the graphical status summary 324a, at the fourth designated position shown, the variant-analysis-status icon 334d indicates a status of the variant analysis of the base-call data, which can be performed by a server (e.g., a local server device). In this example, the variant-analysis-status icon 334d is a circular stop-sign icon representing a stopped status. At the fifth designated position shown in the graphical status summary 324a, the external-transfer-status icon 334e indicates a status of an external-analysis-data transfer data generated during the variant analysis to an external storage. In this example, the external-transfer-status icon 334e is a grey-filled-circle icon representing a not-started status.

As further shown in FIG. 3C, the client device 114 presents a textual-task-status summary for each status icon within the expanded graphical status summary 336a. In particular, the active sequencing interface 314b comprises textual task summaries 338a, 338b, 338c, 338d, and 338e that describe in words a status of the nucleotide sequencing task represented by the run status icon 334a, the external-transfer-status icon 334b, the data-transfer-status icon 334c, the variant-analysis-status icon 334d, and the external-transfer-status icon 334e, respectively. In the expanded graphical status summary 336a, the client device 114 vertically orders the run status icon 334a, the external-transfer-status icon 334b, the data-transfer-status icon 334c, the variant-analysis-status icon 334d, and the external-transfer-status icon 334e from top to bottom and presents the textual task summaries 338a, 338b, 338c, 338d, and 338e, respectively alongside the corresponding status icon.

In addition to the expanded graphical status summary 336a, in some embodiments, the sequencing status system 106 provides more detailed information concerning each nucleotide sequencing task from a taskset for display within the expanded active taskset overview 340a. As shown in FIG. 3C, for instance, the client device 114 presents (i) a start date and time for each nucleotide sequencing task that has been started and (ii) a completed date and time for each nucleotide sequencing task that has been completed.

As further indicated above, in some embodiments, the sequencing status system 106 can intelligently provide a selectable intervention option for a particular nucleotide sequencing task—based on a determined status of the particular nucleotide sequencing task. As shown in FIG. 3C, for instance, the sequencing status system 106 determines a stopped status for a particular nucleotide sequencing task, here, the variant analysis. Based on the determined stopped status, the sequencing status system 106 provides (and the client device 114 presents) a re-initiate option 342a to re-initiate the particular nucleotide sequencing task. Based on receiving an indication of a selection of the re-initiate option 342a from the client device 114, in some cases, the sequencing status system 106 re-initiates the particular nucleotide sequencing task from a point at which the task stopped (e.g., by requeuing the variant analysis to continue). By contrast, in certain embodiments, based on receiving an indication of a selection of the re-initiate option 342a from the client device 114, the sequencing status system 106 re-initiates the particular nucleotide sequencing task from the beginning (e.g., by restarting the variant analysis to continue).

In addition to customizing a selectable intervention option, the sequencing status system 106 can customize a graphical status summary for a nucleotide sequencing taskset. FIG. 3D depicts the sequencing status system 106 generating a customized graphical status summary for a nucleotide sequencing taskset based on the queried status of individual tasks within the nucleotide sequencing taskset. As shown by a transition from FIG. 3B to FIG. 3D—or a transition from FIG. 3C to FIG. 3D—when the client device 114 detects a selection of an expand option 332b, the client device 114 presents an expanded active taskset overview 340b within an active sequencing interface 314c. Upon expansion in FIG. 3D, the active sequencing interface 314c includes both the graphical status summary 324b and an expanded graphical status summary 336b for a same nucleotide sequencing taskset.

As depicted in FIGS. 3B and 3D, the client device 114 presents the graphical status summary 324b with a textual-taskset-status summary 326b as part of the active taskset overview 316b. Because the status of the nucleotide sequencing taskset represented by the graphical status summary 324b differs from the nucleotide sequencing taskset represented by the graphical status summary 324a, the sequencing status system 106 customizes the textual-taskset-status summary 326b to represent the differing status of a corresponding nucleotide sequencing taskset. In some embodiments, the textual-taskset-status summary 326b describes a current status of a last scheduled or last commenced nucleotide sequencing task—here, an external-analysis-data transfer of data (generated during a different variant analysis) to an external storage. In this example, the textual-taskset-status summary 326a indicates a server cannot connect to an external storage.

As further depicted in FIGS. 3B and 3D, the client device 114 presents the graphical status summary 324b with a set of status icons ordered horizontally with respect to the active sequencing interface 314a or 314c. Similar to the graphical status summary 324a, the graphical status summary 324b comprises a run status icon, an external-transfer-status icon, a data-transfer-status icon, a variant-analysis-status icon, and another external-transfer-status icon. But the sequencing status system 106 customizes the set of status icons for the graphical status summary 324b to indicate a status of a different nucleotide sequencing taskset—with a different sequencing run, different variant analysis, and other different tasks. As shown at an external-analysis-data-transfer position (or fifth designated position) within the graphical status summary 324b, for example, an external-transfer-status icon 344 indicates a different status of the external-analysis-data transfer of data resulting from a variant analysis to an external storage. In this example, the client device 114 presents an error icon or warning sign icon to represent an error status for the external-analysis-data transfer.

Similarly, as further depicted in FIG. 3D, the expanded active taskset overview 340b includes the expanded graphical status summary 336b with textual task summaries for status icons differing from the textual task summaries for the expanded graphical status summary 336a depicted in FIG. 3C. In FIG. 3D, for instance, the client device 114 presents a textual-task-status summary 346 that describes the nucleotide sequencing task represented by the external-transfer-status icon 344 with a warning message or an error message. In this example, the textual-task-status summary 346 indicates that the external-analysis-data transfer for the nucleotide sequencing taskset has encountered an error and is unable to complete.

As indicated above, the sequencing status system 106 can likewise customize a selectable intervention option for different nucleotide sequencing tasks-based on a determined status of a particular nucleotide sequencing task. As shown in FIG. 3D, for instance, the sequencing status system 106 determines an error status for the external-analysis-data transfer of data generated during the variant analysis to an external storage. Based on the determined error status, the sequencing status system 106 provides (and the client device 114 presents) a re-initiate option 342b to re-initiate the external-analysis-data transfer. Based on receiving an indication of a selection of the re-initiate option 342b from the client device 114, the sequencing status system 106 re-initiates (or sends a command to re-initiate) the external-analysis-data transfer either from a point at which the transfer encountered an error or from the beginning.

Indeed, the sequencing status system 106 can customize one or both of a graphical status summary for a nucleotide sequencing taskset and a selectable intervention option based on a determined status of the nucleotide sequencing taskset or an individual nucleotide sequencing task. FIG. 3E depicts the sequencing status system 106 generating a customized graphical status summary for a different nucleotide sequencing taskset based on the queried status of individual tasks within the nucleotide sequencing taskset. As shown by a transition from FIG. 3B to FIG. 3E—or a transition from FIG. 3D to FIG. 3E—when the client device 114 detects a selection of an expand option 332c, the client device 114 presents, within an active sequencing interface 314d, the graphical status summary 324c as part of the active taskset overview 316c and an expanded graphical status summary 336c as part of an expanded active taskset overview 340c. Both the graphical status summary 324c and the expanded graphical status summary 336c represent a same nucleotide sequencing taskset.

As depicted in FIG. 3E, the client device 114 presents the graphical status summary 324c with a textual-taskset-status summary 326c as part of the active taskset overview 316c. The sequencing status system 106 customizes the textual-taskset-status summary 326c to represent the differing and current status of its corresponding nucleotide sequencing taskset. As depicted in FIG. 3E, in some embodiments, the textual-taskset-status summary 326c describes as queued for beginning a variant analysis of base-call data from a sequencing run.

As further depicted in FIG. 3E, the client device 114 presents the graphical status summary 324c comprising a run status icon, an external-transfer-status icon, a data-transfer-status icon, a variant-analysis-status icon, and another external-transfer-status icon—each at corresponding designated positions. At a variant-analysis position (or fourth designated position) within the graphical status summary 324c, a variant-analysis-status icon 350 indicates a different status of the variant analysis from other graphical status summaries described above. In this example, the client device 114 presents a light grey clock icon or a lighter shaded clock icon representing a queued status (or a scheduled status) for the variant analysis. Accordingly, as indicated by the variant-analysis-status icon 350, a server or other computing device has yet to commence the variant analysis as part of a corresponding nucleotide sequencing taskset.

Similarly, as further depicted in FIG. 3E, the expanded graphical status summary 336c likewise includes textual task summaries for status icons differing from other textual task summaries for other expanded graphical status summaries described above. For instance, the client device 114 presents a textual-task-status summary 352 that describes the variant analysis represented by the variant-analysis-status icon 350 to indicate that the variant analysis for the nucleotide sequencing taskset is queued or scheduled. In this example, the textual-task-status summary 352, therefore, matches the textual-taskset-status summary 326c. Accordingly, in some cases, a textual-task-status summary for an individual nucleotide sequencing task is the same or similar to a corresponding textual-taskset-status summary for a corresponding nucleotide sequencing taskset.

Similar to the selectable intervention options above, as shown in FIG. 3E, the sequencing status system 106 customizes a selectable intervention option for a particular nucleotide sequencing task. In particular, the sequencing status system 106 determines a queued status or a scheduled status for the variant analysis. Based on the determined error status, the sequencing status system 106 provides (and the client device 114 presents) a cancel option 354 to cancel the variant analysis. Based on receiving an indication of a selection of the cancel option 354 from the client device 114, the sequencing status system 106 cancels the variant analysis or sends a command to cancel the variant analysis.

As noted above, in some embodiments, the sequencing status system 106 provides a textual-taskset-status summary for a nucleotide sequencing taskset that corresponds to a particular nucleotide sequencing task. Indeed, in certain cases, the sequencing status system 106 can customize a textual-taskset-status summary to indicate a subtask for a more particularized status indicator. FIG. 3F depicts an example of a more particularized textual-taskset-status summary. As shown by a transition from FIG. 3B to FIG. 3F—or a transition from FIG. 3E to FIG. 3F—when the client device 114 detects a selection of an expand option 332d, the client device 114 presents, within an active sequencing interface 314e, the graphical status summary 324d as part of the active taskset overview 316d and an expanded graphical status summary 336d as part of an expanded active taskset overview 340d.

As depicted in FIG. 3F, for instance, the client device 114 presents the graphical status summary 324d for a nucleotide sequencing taskset comprising a set of status icons indicating a sequencing run is in progress. In particular, the graphical status summary 324d comprises a run status icon, an external-transfer-status icon, and a data-transfer-status icon each indicating an in-progress status. At each of a sequencing run position (or first designated position), an external-call-data-transfer position (or second designated position), and a data-analysis-transfer position (or third designated position) within the graphical status summary 324d, in this example, the status icon depicts a blue clock icon or a darker shaded clock icon representing an in-progress status. As the graphical status summary 324d thus indicates, a computing device can initiate and perform in parallel multiple nucleotide sequencing tasks within a nucleotide sequencing taskset. The sequencing status system 106 can likewise detect statuses and represent such parallel nucleotide sequencing tasks with corresponding status icons in real time.

While both the graphical status summary 324d and the expanded graphical status summary 336d represent a same nucleotide sequencing taskset, the sequencing status system 106 customizes a textual-taskset-status summary 326d as part of the active taskset overview 316d to include more granular detail. In particular, a sequencing device sends status data for a sequencing cycle to the sequencing status system 106 indicating a particular sequencing cycle and a particular nucleotide-fragment-read type of the sequencing cycle (e.g., most recently detected sequencing cycle and most recently detected nucleotide-fragment-read type). Based on the particularized status data, the sequencing status system 106 provides (and the client device 114 presents) the textual-taskset-status summary 326d to indicate the particular sequencing cycle and the particular nucleotide-fragment-read type. For instance, the textual-taskset-status summary 326d states, “Sequencing: Read 2, Cycle 102/151,” thereby indicating a specific nucleotide-fragment-read type and a specific cycle number.

By contrast, as shown in FIG. 3F, the expanded graphical status summary 336d includes textual task summaries that generally indicate statuses of corresponding nucleotide sequencing tasksets. In particular, the expanded graphical status summary 336d includes a textual-task-status summary for a run status icon, a textual-task-status summary for an external-transfer-status icon, and a textual-task-status summary for a data-transfer-status icon that respectively indicate that a sequencing run, an external-call-data transfer, and a data-analysis transfer are in progress. In other embodiments, however, the sequencing status system 106 can provide textual task summaries in an expanded graphical status summary that include more granular detail.

In addition to the planned sequencing interface 302 and the active sequencing interfaces 314a-314e, in some embodiments, the sequencing status system 106 also provides a completed sequencing interface with summaries of completed nucleotide sequencing tasksets—with, for example, variant analysis completed and data from the variant analysis transferred to an external storage. As shown in FIG. 3G, for instance, the client device 114 presents a completed sequencing interface 356 of the sequencing application 116 within the screen 301. Based on detecting a selection of a completed sequencing tab 358, for example, the client device 114 presents the completed sequencing interface 356.

As further shown in FIG. 3G, the completed sequencing interface 356 includes completed taskset overviews 360 for nucleotide sequencing tasksets that have finished. The completed taskset overviews 360 each in turn include high-level information concerning a completed nucleotide sequencing taskset, including a textual status summary (e.g., Completed), sequencing run name or nucleotide-sequencing-taskset name (e.g., Run 1234), secondary-analysis application type (e.g., DRAGEN Enrichment), collective sequencing metrics, and date completed.

In addition to providing graphical user interfaces that summarize nucleotide sequencing tasksets, in some embodiments, the sequencing status system 106 provides graphical user interfaces comprises details concerning nucleotide sequencing tasksets. FIGS. 4A-4B illustrate the client device 114 presenting graphical user interfaces comprising details for a selected nucleotide sequencing taskset. Based on detecting a selection of a sequencing run name or a nucleotide-sequencing-taskset name in one of the graphical user interfaces shown in FIGS. 3A-3G, for example, the client device 114 presents a sequencing details interface 404a within a screen 402, as shown in FIG. 4A.

As shown in FIG. 4A, the client device 114 provides taskset materials details 406, sequencing run details 408, and sample sequencing metrics 410 for display within the sequencing details interface 404a. By providing the taskset materials details 406, the sequencing status system 106 provides, for display in the sequencing details interface 404a, a sequencing run name or nucleotide-sequencing-taskset name (e.g., Run 234), a sequencing run description or nucleotide-sequencing-taskset description, a secondary-analysis application type (e.g., DRAGEN Enrichment) and version (e.g., 3.5.2), a sequencing software application version (e.g., 2.0.5), an owner, and a textual-taskset-status summary (e.g., Completed).

As further shown in FIG. 4A, by providing the sequencing run details 408, the sequencing status system 106 provides, for display in the sequencing details interface 404a, a sequencing device name (e.g., Sequencer 2021), a technician name, a sample-nucleotide-slide type (e.g., S4 for a flow cell), a nucleotide-fragment read type for the sequencing run (e.g., Paired End), and nucleotide-fragment read lengths (e.g., Read 1: 151, index 1: 10).

As further shown in FIG. 4A, by providing the sample sequencing metrics 410, the sequencing status system 106 provides, for display in the sequencing details interface 404a, collective sequencing metrics specific to a sample. As organize by sample number from a nucleotide sequencing taskset, the sample sequencing metrics 410 include a total number of nucleotide-fragment reads passing a quality filter, a collective base-call-quality metrics for different nucleotide-fragment-read type (e.g., Read 1%>=Q30), and callability metrics.

From within the sequencing details interface 404a, in some embodiments, the sequencing status system 106 provides a selectable status option 412 that, when selected, causes the client device 114 to show a graphical status summary for the relevant nucleotide sequencing taskset detailed in the sequencing details interface 404a. As shown in FIG. 4B, based on detecting a selection of the selectable status option 412, for instance, the client device 114 presents an expanded graphical status summary 414 of the relevant nucleotide sequencing taskset within a sequencing details interface 404b. In this example, the client device 114 also presents textual-task-status summaries, start dates, and completed dates alongside (or in line with) status icons within the expanded graphical status summary 414.

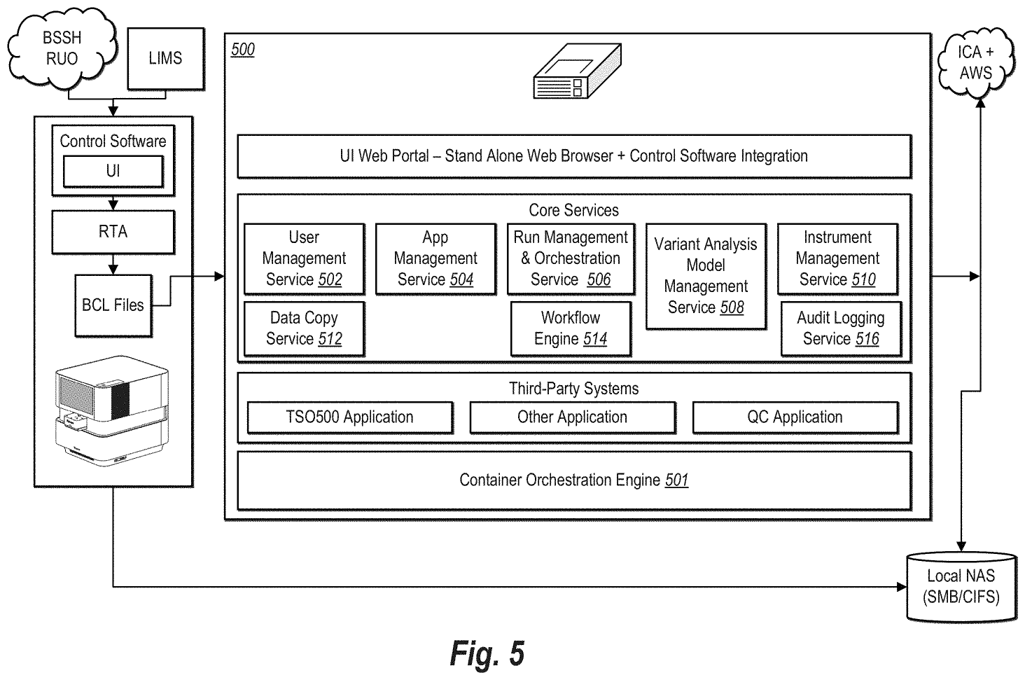

In certain embodiments, the sequencing system 104 utilizes containers and pods to execute external workflows associated with nucleotide reads and base calls of a sample sequence. In particular, the sequencing system 104 can analyze sequencing data via a diagnostic workflow to identify genetic markers or hereditary traits indicated within a genomic sample. The sequencing status system 106 can send or receive data indicating a status of sequencing tasks performed by such containers or pods. FIG. 5 illustrates an example diagram of components, applications, devices, and containers of a system architecture (e.g., installed on a local server device) involved in implementing an external diagnostic workflow in accordance with one or more embodiments.

As illustrated in FIG. 5, the sequencing system 104 utilizes a sequencing device (e.g., the sequencing device 108) to communicate with various components or systems and to perform sequencing operations used by, and/or as instructed by, a version of a variant analysis model on a local server (e.g., a version of DRAGEN on the local server device 102). For example, the sequencing system 104 communicates with a BaseSpace Sequencing Hub (“BSSH”) or cloud-based interface for research use only (“RUO”) and a lab information management system (“LIMS”) to generate base calls and other sequencing data for nucleotide bases of a genomic sample.

Based on information from the BSSH RUO and/or the LIMS, the sequencing system 104 performs a real-time analysis (“RTA”) of a sample. More specifically, the sequencing system 104 performs RTA to determine base calls, variant calls, and/or various metrics from nucleotide bases of a genomic sample according to a sequencing plan. Based on the RTA, the sequencing system 104 generates a binary base call (“BCL”) file that includes raw data generated and output by one or more sequencing runs (e.g., via the RTA). Indeed, the BCL file can indicate base calls, variant calls, and/or other sequencing information for interpretation by a variant analysis model and/or some other system.

To organize or plan a sequencing run of the RTA, the sequencing system 104 provides control software (e.g., including a user interface) for planning or scheduling a sequencing run on a particular sample. Indeed, the sequencing system 104 provides control software and a user interface for planning one or more sequencing runs to, for example, test a genomic sample for a particular genetic marker according to plan parameters. For instance, the control software enables a user to specify parameters for a sequencing run and/or to test for specific markers. As shown, the sequencing system 104 can integrate the control software for the sequencing device with a user interface web portal (which includes a standalone web browser and control software integration) to interface with the sequencing device for planning a sequencing run.

In some cases, the sequencing system 104 facilitates local planning for a sequencing run, where the planning software (e.g., the control software) is hosted by a local server device, such as a local edge server. In these or other cases, the sequencing system 104 facilitates cloud planning for a sequencing run, where the planning software (e.g., the control software) is hosted on a cloud server rather than a local server. In a similar fashion, the execution of a variant analysis model can be local or cloud-based as well, depending on whether the server hosting the variant analysis model is a local server (e.g., the local server device 102). Accordingly, (i) the sequencing status system 106 can be executed either locally on a local server device located at or near the sequencing device 108 or remotely on a cloud-based server device in combination with (ii) a variant analysis model (e.g., DRAGEN) executed either locally on a local server device located at or near the sequencing device 108 or remotely on a cloud-based server device.

As further illustrated in FIG. 5, the system architecture 500 of the sequencing system 104 includes, or communicates with, containers or systems associated with one or more core services. Indeed, as shown, the sequencing system 104 includes the services of the system architecture 500. To manage or orchestrate the various services of the system architecture 500, the system architecture 500 includes a container orchestration engine 501 (e.g., K3S or Kubernetes) to manage and implement various pods and containers associated with performing genomic analyses via diagnostic workflows. As described, the sequencing system 104 utilizes a container orchestration engine 501 to orchestrate or coordinate a diagnostic workflow to analyze a genomic sequence for base calling, variant calling (e.g., as indicated by an application of a third-party system). The container orchestration engine 501 includes pods and containers to perform other functions as well, including user management, application management, run management, variant analysis model management, instrument management, data copying, and audit logging.

For example, the system architecture 500 includes a user management service 502 that includes a set of one or more user management pods or containers. The user management service 502 performs various processes or functions for providing a single sign-on (“SSO”) experience system wide. Specifically, the user management service 502 can include one or more containers or pods that include or access user information for a third-party system to, for example, determine a diagnostic workflow (e.g., from one of the third-party systems) for analyzing a genomic sequence, including user settings or preferences for executing the diagnostic workflow. Based on the determination of the diagnostic workflow and/or the user settings, the user management service 502 can communicate with other services of the system architecture 500 to initiate performance of the diagnostic workflow to analyze a genomic sequence accordingly.

In addition, the system architecture 500 includes or utilizes an application management service 504 in communication with the container orchestration engine 501. For example, the application management service 504 manages application package installation for diagnostic workflows. In some cases, the application management service 504 further includes a resource manager. The resource manager can access or utilize a genomic analysis device resource as specified by an application specification and/or as part of a diagnostic workflow. To elaborate, the resource manager identifies a resource label to access a designated resource, such as an FPGA or a CPU, as a schedulable resource for access via the container orchestration engine. Indeed, in some cases, the application management service 504 includes (or receives from a third-party system) an application specification that indicates an FPGA or a CPU or some other genomic analysis device for executing a diagnostic workflow of a genomic analysis application (or a particular workflow pod), and the resource manager therefore accesses or communicates with the specified device (or other resource) for facilitating execution of the genomic analysis application (or the particular workflow pod).

As further shown, the system architecture 500 includes or utilizes a run management and orchestration service 506. To elaborate, the run management and orchestration service 506 includes one or more containers or pods for facilitating and executing genomic analysis via a diagnostic workflow, such as a sequencing run, a primary analysis, a secondary analysis, or a tertiary analysis. Indeed, the run management and orchestration service 506 includes computer code or instructions for executing a sequencing run (and/or further analysis) according to an installed version of a variant analysis model. For instance, the run management and orchestration service 506 communicates with the workflow engine 514 to execute a custom diagnostic workflow for an application, such as an application associated with a third-party system (e.g., an oncology assay application, such as TSO500 application; a QC application; or another application). The run management and orchestration service 506 further includes code for communicating with the data copy service 512 to copy input and output sequencing data (e.g., from a BCL file generated by a sequencing device) for performing a genomic analysis and/or for storing in a database, such as a local network attached storage (“NAS”), server message block (“SMB”), or common internet file system (“CIFS”). In some embodiments, the sequencing status system 106 is part of the run management and orchestration service 506.

In addition, the system architecture 500 includes a variant analysis model management service 508. In particular, the variant analysis model management service 508 includes one or more containers or pods for managing a variant analysis model for performing genomic analysis. For example, the variant analysis model management service 508 implements a particular diagnostic workflow using a variant analysis model to detect a genetic marker for a certain condition within a sample genomic sequence. In addition, the variant analysis model management service 508 manages model peripherals, such as licensing, self-testing, and version authentication for a variant analysis model.

As further illustrated in FIG. 5, the system architecture 500 includes an instrument management service 510. In one or more embodiments, the instrument management service 510 includes one or more containers or pods for pairing and monitoring instruments used as part of a sequencing workflow and/or a genomic analysis workflow after sequencing. For instance, the instrument management service 510 manages instruments of a sequencing device and/or a variant analysis model to pair compatible instruments with indicated versions of a variant analysis model (or vice-versa). The system architecture 500 further includes an audit logging service 516 for monitoring and logging performance of instruments, components of a variant analysis model, and/or containers within an application workflow. For instance, the audit logging service 516 detects and logs errors or other auditing information associated with the system architecture 500.

As suggested above in the description of FIG. 1 and elsewhere, using the system architecture 500 illustrated in FIG. 5, the sequencing status system 106 can be deployed locally on an edge server (e.g., the local server device 102) or in the cloud such as on cloud-based servers hosting Illumina Connected Analytics (“ICA”) and/or cloud-based servers from Amazon Web Services (“AWS”). For example, the sequencing status system 106 can be executed locally on the local server device 102—or remotely on the server device(s) 110—as part of planning software that plans resources based on user input for sequencing runs or other assays, and a variant analysis model from the sequencing system 104 can likewise be executed locally on the local server device 102—or remotely on the server device(s) 110—to analyze BCL data and determine variant calls or other metrics.

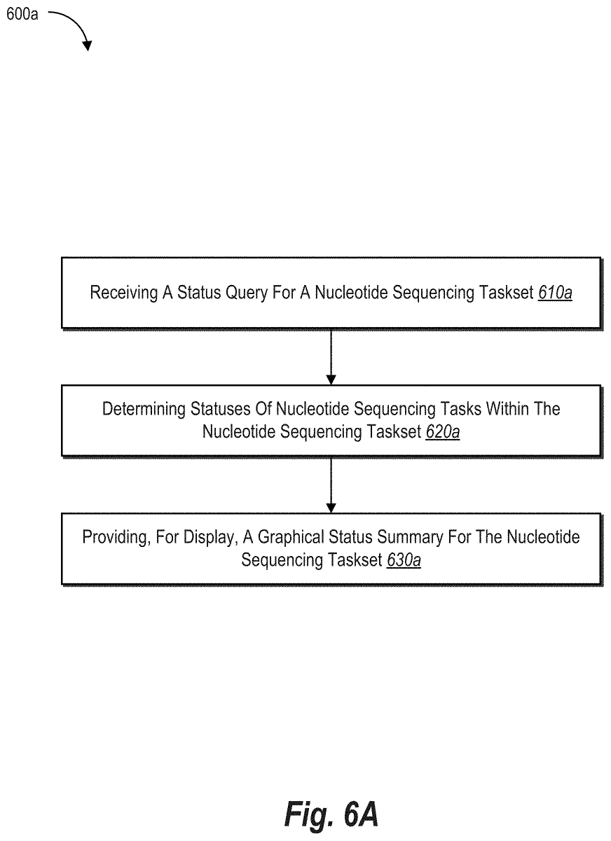

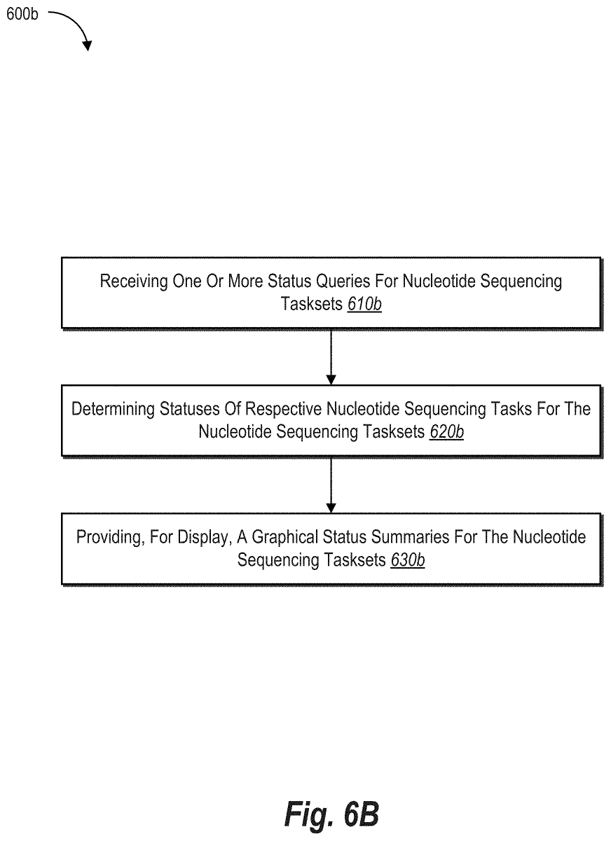

Turning now to FIG. 6A, this figure illustrates a flowchart of a series of acts 600a of querying the status of various tasks of a nucleotide sequencing taskset and generating a graphical status summary for the nucleotide sequencing taskset in accordance with one or more embodiments of the present disclosure. While FIG. 6A illustrates acts according to one embodiment, alternative embodiments may omit, add to, reorder, and/or modify any of the acts shown in FIG. 6A. The acts of FIG. 6A can be performed as part of a method. Alternatively, a non-transitory computer readable storage medium can comprise instructions that, when executed by one or more processors, cause a computing device or a system to perform the acts depicted in FIG. 6A. In still further embodiments, a system comprising at least one processor and a non-transitory computer readable medium comprising instructions that, when executed by one or more processors, cause the system to perform the acts of FIG. 6A.

As shown in FIG. 6A, the acts 600a include an act 610a of receiving a status query for a nucleotide sequencing taskset. In particular, in some embodiments, the act 610a includes receiving, from a computing device, a status query for a nucleotide sequencing taskset depicted by an active sequencing interface.

As further shown in FIG. 6A, the acts 600a include an act 620a of determining statuses of nucleotide sequencing tasks within the nucleotide sequencing taskset. In particular, in some embodiments, the act 620a includes determining, for the nucleotide sequencing taskset, statuses of a sequencing run, a data-analysis transfer of base-call data generated during the sequencing run, and a variant analysis of the base-call data. Further, in some cases, the act 620a includes determining, for the nucleotide sequencing taskset and from a sequencing device, a status of a sequencing run; and determining, for the nucleotide sequencing taskset and from a local server corresponding to the sequencing device, statuses of a data-analysis transfer of base-call data generated during the sequencing run and a variant analysis of the base-call data. In some cases, determining a status of the data-analysis transfer comprises determining a status of a transfer of base-call data generated during the sequencing run from the sequencing device to the local server.

As suggested above, in certain embodiments, the act 620a includes determining a status of an external-call-data transfer of the base-call data generated during the sequencing run to an external storage; and providing the graphical status summary further comprising an external-transfer-status icon indicating a status of the external-call-data transfer. Similarly, in some cases, the act 620a includes determining a status of an external-analysis-data transfer of data generated during the variant analysis to an external storage; and providing the graphical status summary further comprising an external-transfer-status icon indicating a status of the external-analysis-data transfer.

As further shown in FIG. 6A, the acts 600a include an act 630a of providing for display a graphical status summary for the nucleotide sequencing taskset. In particular, in certain implementations, the act 630a includes providing, for display within the active sequencing interface, a graphical status summary for the nucleotide sequencing taskset comprising a run status icon indicating a status of the sequencing run, a data-transfer-status icon indicating a status of the data-analysis transfer, and a variant-analysis-status icon indicating a status of the variant analysis. In some cases, the act 630a includes providing the graphical status summary for display on the sequencing device, local computing device connected to a local server, or an external computing device connected to the server via a network.

In addition to the acts 610a-630a, in certain implementations, the acts 600a further include determining a collective base-call-quality metric indicating an accuracy of base calls generated during the sequencing run; and providing, for display within the active sequencing interface, the collective base-call-quality metric. In some cases, the collective base-call-quality metric is displayed proximate to the graphical status summary. Similarly, in some embodiments, the acts 600a further include determining a collective pass filter metric indicating a subset of base calls generated during the sequencing run that satisfy a quality filter; and providing, for display within the active sequencing interface, the collective pass filter metric. In some cases, the collective pass filter metric is displayed proximate to the graphical status summary.

As further suggested above, in certain cases, the acts 600a further include determining an updated status of one or more of the sequencing run, the data-analysis transfer, or the variant analysis; and providing an updated graphical status summary comprising one or more of an updated run status icon indicating an updated status of the sequencing run, an updated data-transfer-status icon indicating an updated status of the data-analysis transfer, and an updated variant-analysis-status icon indicating an updated status of the variant analysis.

Beyond or in the alternative to the acts 600a described above, in some embodiments, the acts 600a include receiving an indication of a user selection of an expand option corresponding to the graphical status summary; and based on the indication of the user selection of the expand option, providing, for display within the active sequencing interface, an expanded graphical status summary comprising a textual status summary for the run status icon, a textual status summary for the data-transfer-status icon, and a textual status summary for the variant-analysis-status icon.

As described above, in certain implementations, the acts 600a include receiving an indication of a user selection of a cancel option corresponding to one or more of the sequencing run, the data-analysis transfer, or the variant analysis; and based on the indication of the user selection of the cancel option, canceling one or more of the sequencing run, the data-analysis transfer, or the variant analysis. Similarly, in some cases, the acts 600a include receiving an indication of a user selection of a re-initiate option corresponding to one or more of the sequencing run, the data-analysis transfer, or the variant analysis; and based on the indication of the user selection of the re-initiate option, re-initiating one or more of the sequencing run, the data-analysis transfer, or the variant analysis.

Turning now to FIG. 6B, this figure illustrates a flowchart of a series of acts 600b of querying the status of various tasks of nucleotide sequencing tasksets and generating graphical status summaries for the nucleotide sequencing tasksets in accordance with one or more embodiments of the present disclosure. While FIG. 6B illustrates acts according to one embodiment, alternative embodiments may omit, add to, reorder, and/or modify any of the acts shown in FIG. 6B. The acts of FIG. 6B can be performed as part of a method. Alternatively, a non-transitory computer readable storage medium can comprise instructions that, when executed by one or more processors, cause a computing device or a system to perform the acts depicted in FIG. 6B. In still further embodiments, a system comprising at least one processor and a non-transitory computer readable medium comprising instructions that, when executed by one or more processors, cause the system to perform the acts of FIG. 6B.

As shown in FIG. 6B, the acts 600b include an act 610b of receiving one or more status queries for nucleotide sequencing tasksets. In particular, in some embodiments, the act 610b includes receiving, from a computing device, one or more status queries for nucleotide sequencing tasksets depicted by an active sequencing interface.

As further shown in FIG. 6B, the acts 600b include an act 620b of determining statuses of respective nucleotide sequencing tasks for the nucleotide sequencing tasksets. In particular, in some embodiments, the act 620b includes determining, for the nucleotide sequencing tasksets, statuses of respective sequencing runs, respective data-analysis transfers of base-call data generated during the respective sequencing runs, and respective variant analyses of the base-call data.

For instance, in some cases, the act 620b includes determining, for a first nucleotide sequencing taskset among the nucleotide sequencing tasksets, statuses of a first sequencing run, a first data-analysis transfer of base-call data generated during the first sequencing run, and a first variant analysis of the base-call data; and determining, for a second nucleotide sequencing taskset among the nucleotide sequencing tasksets, statuses of a second sequencing run, a second data-analysis transfer of base-call data generated during the second sequencing run, and a second variant analysis of the base-call data.

As further shown in FIG. 6B, the acts 600b include an act 630b of providing for display graphical status summaries for the nucleotide sequencing tasksets. In particular, in certain implementations, the act 630b includes providing, for display within the active sequencing interface, graphical status summaries for the nucleotide sequencing tasksets comprising run status icon indicating statuses of the respective sequencing runs, data-transfer-status icons indicating statuses of the respective data-analysis transfers, and variant-analysis-status icons indicating statuses of the respective variant analyses.

In some cases of the act 630b, providing the graphical status summaries for the nucleotide sequencing tasksets comprises providing, for display within the active sequencing interface: a first graphical status summary for the first nucleotide sequencing taskset comprising a first run status icon indicating a status of the first sequencing run, a first data-transfer-status icon indicating a status of the first data-analysis transfer, and a first variant-analysis-status icon indicating a status of the first variant analysis; and a second graphical status summary for the second nucleotide sequencing taskset comprising a second run status icon indicating a status of the second sequencing run, a second data-transfer-status icon indicating a status of the second data-analysis transfer, and a second variant-analysis-status icon indicating a status of the second variant analysis.

In addition to the acts 610b-630b, in certain implementations, the acts 600b further include determining statuses of respective external-call-data transfers of the base-call data generated during the respective sequencing runs to an external storage; and providing the graphical status summaries further comprising external-transfer-status icons indicating statuses of the respective external-call-data transfers. Similarly, in some cases, the acts 600b further include determining statuses of respective external-analysis-data transfers of data generated during the respective variant analyses to an external storage; and providing the graphical status summaries further comprising external-transfer-status icons indicating statuses of the respective external-analysis-data transfers.

As further suggested above, in certain cases, the acts 600b further include receiving an indication of a user selection of an expand option corresponding to a graphical status summary among the graphical status summaries; and based on the indication of the user selection of the expand option, provide, for display within the active sequencing interface, an expanded graphical status summary comprising a textual status summary for a run status icon within the graphical status summary, a textual status summary for a data-transfer-status icon within the graphical status summary, and a textual status summary for a variant-analysis-status icon within the graphical status summary.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a target nucleic acid (i.e., a nucleic-acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, Conn., a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, Calif.) and devices described in U.S. Ser. No. 13/273,666, which is incorporated herein by reference.

The sequencing system described above sequences nucleic-acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the sequencing status system 106 can include software, hardware, or both. For example, the components of the sequencing status system 106 can include one or more instructions stored on a computer-readable storage medium and executable by processors of one or more computing devices (e.g., the client device 114). When executed by the one or more processors, the computer-executable instructions of the sequencing status system 106 can cause the computing devices to perform the bubble detection methods described herein. Alternatively, the components of the sequencing status system 106 can comprise hardware, such as special purpose processing devices to perform a certain function or group of functions. Additionally, or alternatively, the components of the sequencing status system 106 can include a combination of computer-executable instructions and hardware.

Furthermore, the components of the sequencing status system 106 performing the functions described herein with respect to the sequencing status system 106 may, for example, be implemented as part of a stand-alone application, as a module of an application, as a plug-in for applications, as a library function or functions that may be called by other applications, and/or as a cloud-computing model. Thus, components of the sequencing status system 106 may be implemented as part of a stand-alone application on a personal computing device or a mobile device. Additionally, or alternatively, the components of the sequencing status system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 7 illustrates a block diagram of a computing device 700 that may be configured to perform one or more of the processes described above. One will appreciate that one or more computing devices such as the computing device 700 may implement the sequencing status system 106 and the sequencing system 104. As shown by FIG. 7, the computing device 700 can comprise a processor 702, a memory 704, a storage device 706, an I/O interface 708, and a communication interface 710, which may be communicatively coupled by way of a communication infrastructure 712. In certain embodiments, the computing device 700 can include fewer or more components than those shown in FIG. 7. The following paragraphs describe components of the computing device 700 shown in FIG. 7 in additional detail.

In one or more embodiments, the processor 702 includes hardware for executing instructions, such as those making up a computer program. As an example, and not by way of limitation, to execute instructions for dynamically modifying workflows, the processor 702 may retrieve (or fetch) the instructions from an internal register, an internal cache, the memory 704, or the storage device 706 and decode and execute them. The memory 704 may be a volatile or non-volatile memory used for storing data, metadata, and programs for execution by the processor(s). The storage device 706 includes storage, such as a hard disk, flash disk drive, or other digital storage device, for storing data or instructions for performing the methods described herein.

The I/O interface 708 allows a user to provide input to, receive output from, and otherwise transfer data to and receive data from computing device 700. The I/O interface 708 may include a mouse, a keypad or a keyboard, a touch screen, a camera, an optical scanner, network interface, modem, other known I/O devices or a combination of such I/O interfaces. The I/O interface 708 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 708 is configured to provide graphical data to a display for presentation to a user. The graphical data may be representative of one or more graphical user interfaces and/or any other graphical content as may serve a particular implementation.

The communication interface 710 can include hardware, software, or both. In any event, the communication interface 710 can provide one or more interfaces for communication (such as, for example, packet-based communication) between the computing device 700 and one or more other computing devices or networks. As an example, and not by way of limitation, the communication interface 710 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 710 may facilitate communications with various types of wired or wireless networks. The communication interface 710 may also facilitate communications using various communication protocols. The communication infrastructure 712 may also include hardware, software, or both that couples components of the computing device 700 to each other. For example, the communication interface 710 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

As used herein, the term “object” includes all things that are suitable for imaging, viewing, analyzing, inspecting, or profiling with the optical systems described herein. By way of example only, objects may include semiconductor wafers or chips, recordable media, samples, flow cells, microparticles, slides, or microarrays. Objects generally include one or more surfaces and/or one or more interfaces that a user may desire to image, view, analyze, inspect, and/or determine a profile thereof. The objects may have surfaces or interfaces with relief features such as wells, pits, ridges, bumps, beads or the like.

As indicated above in the description of a “sample,” a sample may be imaged or scanned for subsequent analysis. In particular embodiments, a sample may include biological or chemical substances of interests and, optionally, an optical substrate that supports the biological or chemical substances. As such, a sample may or may not include an optical substrate. As used herein, the term “biological or chemical substances” is not intended to be limiting, but may include a variety of biological or chemical substances that are suitable for being imaged or examined with the optical systems described herein. For example, biological or chemical substances include biomolecules, such as nucleosides, nucleic acids, polynucleotides, oligonucleotides, proteins, enzymes, polypeptides, antibodies, antigens, ligands, receptors, polysaccharide, carbohydrate, polyphosphates, nanopores, organelles, lipid layers, cells, tissues, organisms, and biologically active chemical compound(s) such as analogs or mimetics of the aforementioned species.

The biological or chemical substances may be supported by an optical substrate. As used herein, the term “optical substrate” is not intended to be limiting, but may include various materials that support the biological or chemical substances and permit the biological or chemical substances to be at least one of viewed, imaged, and examined. For example, the optical substrate may comprise a transparent material that reflects a portion of incident light and refracts a portion of the incident light. Alternatively, the optical substrate may be, for example, a mirror that reflects the incident light entirely such that no light is transmitted through the optical substrate. Typically, the optical substrate has a flat surface. However, the optical substrate can have a surface with relief features such as wells, pits, ridges, bumps, beads or the like.

In an exemplary embodiment, the optical substrate is a flow cell having flow channels where nucleic acids are sequenced. However, in alternative embodiments, the optical substrate may include one or more slides, planar chips (such as those used in microarrays), or microparticles. In such cases where the optical substrate includes a plurality of microparticles that support the biological or chemical substances, the microparticles may be held by another optical substrate, such as a slide or grooved plate. In particular embodiments, the optical substrate includes diffraction grating based encoded optical identification elements similar to or the same as those described in pending U.S. patent application Ser. No. 10/661,234, entitled Diffraction Grating Based Optical Identification Element, filed Sep. 12, 2003, which is incorporated herein by reference in its entirety, discussed more hereinafter. A bead cell or plate for holding the optical identification elements may be similar to or the same as that described in pending U.S. patent application Ser. No. 10/661,836, entitled “Method and Apparatus for Aligning Microbeads in Order to Interrogate the Same”, filed Sep. 12, 2003, and U.S. Pat. No. 7,164,533, entitled “Hybrid Random Bead/Chip Based Microarray”, issued Jan. 16, 2007, as well as U.S. patent application Ser. No. 60/609,583, entitled “Improved Method and Apparatus for Aligning Microbeads in Order to Interrogate the Same”, filed Sep. 13, 2004, Ser. No. 60/1,010,910, entitled “Method and Apparatus for Aligning Microbeads in Order to Interrogate the Same”, filed Sep. 17, 2004, each of which is incorporated herein by reference in its entirety.

As used herein, the term “optical components” or “focus components” includes various elements that affect the transmission of light. Optical components may be, for example, reflectors, dichroics, beam splitters, collimators, lenses, filters, wedges, prisms, mirrors, and the like.

By way of example, optical systems described herein may be constructed to include various components and assemblies as described in PCT application PCT/US07/07991, entitled “System and Devices for Sequence by Synthesis Analysis”, filed Mar. 30, 2007 and/or to include various components and assemblies as described in PCT application PCT/US2008/077850, entitled “Fluorescence Excitation and Detection System and Method”, filed Sep. 26, 2008, both of which the complete subject matter are incorporated herein by reference in their entirety. In particular embodiments, optical systems can include various components and assemblies as described in U.S. Pat. No. 7,329,860, of which the complete subject matter is incorporated herein by reference in its entirety. Optical systems can also include various components and assemblies as described in U.S. patent application Ser. No. 12/638,770, filed on Dec. 15, 2009, of which the complete subject matter is incorporated herein by reference in its entirety.

In particular embodiments, methods, and optical systems described herein may be used for sequencing nucleic acids. For example, sequencing-by-synthesis (SBS) protocols are particularly applicable. In SBS, a plurality of fluorescently labeled modified nucleotides are used to sequence dense clusters of amplified DNA (possibly millions of clusters) present on the surface of an optical substrate (e.g., a surface that at least partially defines a channel in a flow cell). The flow cells may contain nucleic acid samples for sequencing where the flow cells are placed within the appropriate flow cell holders. The samples for sequencing can take the form of single nucleic acid molecules that are separated from each other so as to be individually resolvable, amplified populations of a nucleic acid molecules in the form of clusters or other features, or beads that are attached to one or more molecules of nucleic acid. The nucleic acids can be prepared such that they comprise an oligonucleotide primer adjacent to an unknown target sequence. To initiate the first SBS sequencing cycle, one or more differently labeled nucleotides, and DNA polymerase, etc., can be flowed into/through the flow cell by a fluid flow subsystem (not shown). Either a single type of nucleotide can be added at a time, or the nucleotides used in the sequencing procedure can be specially designed to possess a reversible termination property, thus allowing each cycle of the sequencing reaction to occur simultaneously in the presence of several types of labeled nucleotides (e.g., A, C, T, G). The nucleotides can include detectable label moieties such as fluorophores. Where the four nucleotides are mixed together, the polymerase is able to select the correct base to incorporate and each sequence is extended by a single base. One or more lasers may excite the nucleic acids and induce fluorescence. The fluorescence emitted from the nucleic acids is based upon the fluorophores of the incorporated base, and different fluorophores may emit different wavelengths of emission light. Exemplary sequencing methods are described, for example, in Bentley et al., Nature 456:53-59 (2008), WO 04/018497; U.S. Pat. No. 7,057,026; WO 91/06678; WO 07/123,744; U.S. Pat. Nos. 7,329,492; 7,211,414; 7,315,019; 7,405,281, and US 2008/0108082, each of which is incorporated herein by reference.

Other sequencing techniques that are applicable for use of the methods and systems set forth herein are pyrosequencing, nanopore sequencing, and sequencing by ligation. Exemplary pyrosequencing techniques and samples that are particularly useful are described in U.S. Pat. Nos. 6,210,891; 6,258,568; 6,274,320 and Ronaghi, Genome Research 11:3-11 (2001), each of which is incorporated herein by reference. Exemplary nanopore techniques and samples that are also useful are described in Deamer et al., Acc. Chem. Res. 35:817-825 (2002); Li et al., Nat. Mater. 2:611-615 (2003); Soni et al., Clin Chem. 53:1996-2001 (2007) Healy et al., Nanomed. 2:459-481 (2007) and Cockroft et al., J. am. Chem. Soc. 130:818-820; and U.S. Pat. No. 7,001,792, each of which is incorporated herein by reference. Any of a variety of samples can be used in these systems such as substrates having beads generated by emulsion PCR, substrates having zero-mode waveguides, substrates having biological nanopores in lipid bilayers, solid-state substrates having synthetic nanopores, and others known in the art. Such samples are described in the context of various sequencing techniques in the references cited above and further in US 2005/0042648; US 2005/0079510; US 2005/0130173; and WO 05/010145, each of which is incorporated herein by reference.

In other embodiments, optical systems described herein may be utilized for detection of samples that include microarrays. A microarray may include a population of different probe molecules that are attached to one or more substrates such that the different probe molecules can be differentiated from each other according to relative location. An array can include different probe molecules, or populations of the probe molecules, that are each located at a different addressable location on a substrate. Alternatively, a microarray can include separate optical substrates, such as beads, each bearing a different probe molecule, or population of the probe molecules, that can be identified according to the locations of the optical substrates on a surface to which the substrates are attached or according to the locations of the substrates in a liquid. Exemplary arrays in which separate substrates are located on a surface include, without limitation, a Sentrix® Array or Sentrix® BeadChip Array available from Illumina®, Inc. (San Diego, Calif.) or others including beads in wells such as those described in U.S. Pat. Nos. 6,266,459, 6,355,431, 6,770,441, and 6,859,570; and PCT Publication No. WO 00/63437, each of which is hereby incorporated by reference. Other arrays having particles on a surface include those set forth in US 2005/0227252; WO 05/033681; and WO 04/024328, each of which is hereby incorporated by reference.

Any of a variety of microarrays known in the art, including, for example, those set forth herein, can be used in embodiments of the invention. A typical microarray contains sites, sometimes referred to as features, each having a population of probes. The population of probes at each site is typically homogenous having a single species of probe, but in some embodiments the populations can each be heterogeneous. Sites or features of an array are typically discrete, being separated with spaces between each other. The size of the probe sites and/or spacing between the sites can vary such that arrays can be high density, medium density or lower density. High density arrays are characterized as having sites separated by less than about 15 μm. Medium density arrays have sites separated by about 15 to 30 μm, while low density arrays have sites separated by greater than 30 μm. An array useful in the invention can have sites that are separated by less than 100 μm, 50 μm, 10 μm, 5 μm, 1 μm, or 0.5 μm. An apparatus or method of an embodiment of the invention can be used to image an array at a resolution sufficient to distinguish sites at the above densities or density ranges.

Further examples of commercially available microarrays that can be used include, for example, an Affymetrix® GeneChip® microarray or other microarray synthesized in accordance with techniques sometimes referred to as VLSIPS™ (Very Large Scale Immobilized Polymer Synthesis) technologies as described, for example, in U.S. Pat. Nos. 5,324,633; 5,744,305; 5,451,683; 5,482,867; 5,491,074; 5,624,711; 5,795,716; 5,831,070; 5,856,101; 5,858,659; 5,874,219; 5,968,740; 5,974,164; 5,981,185; 5,981,956; 6,025,601; 6,033,860; 6,090,555; 6,136,269; 6,022,963; 6,083,697; 6,291,183; 6,309,831; 6,416,949; 6,428,752 and 6,482,591, each of which is hereby incorporated by reference. A spotted microarray can also be used in a method according to an embodiment of the invention. An exemplary spotted microarray is a CodeLink™ Array available from Amersham Biosciences. Another microarray that is useful is one that is manufactured using inkjet printing methods such as SurePrint™ Technology available from Agilent Technologies.

The systems and methods set forth herein can be used to detect the presence of a particular target molecule in a sample contacted with the microarray. This can be determined, for example, based on binding of a labeled target analyte to a particular probe of the microarray or due to a target-dependent modification of a particular probe to incorporate, remove, or alter a label at the probe location. Any one of several assays can be used to identify or characterize targets using a microarray as described, for example, in U.S. Patent Application Publication Nos. 2003/0108867; 2003/0108900; 2003/0170684; 2003/0207295; or 2005/0181394, each of which is hereby incorporated by reference.

Exemplary labels that can be detected in accordance with embodiments of the invention, for example, when present on a microarray include, but are not limited to, a chromophore; luminophore; fluorophore; optically encoded nanoparticles; particles encoded with a diffraction-grating; electrochemiluminescent label such as Ru(bpy)³²⁺; or moiety that can be detected based on an optical characteristic. Fluorophores that may be useful include, for example, fluorescent lanthanide complexes, including those of Europium and Terbium, fluorescein, rhodamine, tetramethylrhodamine, eosin, erythrosin, coumarin, methyl-coumarins, pyrene, Malacite green, Cy3, Cy5, stilbene, Lucifer Yellow, Cascade Blue™, Texas Red, alexa dyes, phycoerythin, bodipy, and others known in the art such as those described in Haugland, Molecular Probes Handbook, (Eugene, Oreg.) 6th Edition; The Synthegen catalog (Houston, Tex.), Lakowicz, Principles of Fluorescence Spectroscopy, 2nd Ed., Plenum Press New York (1999), or WO 98/59066, each of which is hereby incorporated by reference.

In particular embodiments, the optical system can be configured for Time Delay Integration (TDI) for example in line scanning embodiments as described, for example, in U.S. Pat. No. 7,329,860, of which the complete subject matter is incorporated herein by reference in its entirety. By way of example, the optical assembly may have a 0.75 NA lens and a focus accuracy of +/−125 to 500 nm. The resolution can be 50 to 100 nm. The system may be able to obtain 1,000-10,000 measurements/second unfiltered.

Although embodiments are exemplified with regard to detection of samples that includes biological or chemical substances supported by an optical substrate, it will be understood that other samples can be analyzed, examined, or imaged by the embodiments described herein. Other exemplary samples include, but are not limited to, biological specimens such as cells or tissues, electronic chips such as those used in computer processors, or the like. Examples of some of the applications include microscopy, satellite scanners, high-resolution reprographics, fluorescent image acquisition, analyzing and sequencing of nucleic acids, DNA sequencing, sequencing-by-synthesis, imaging of microarrays, imaging of holographically encoded microparticles and the like.

In other embodiments, the optical systems may be configured to inspect an object to determine certain features or structures of the object. For example, the optical systems may be used to inspect a surface of the object, (e.g., semiconductor chip, silicon wafer) to determine whether there are any deviations or defects on the surface.

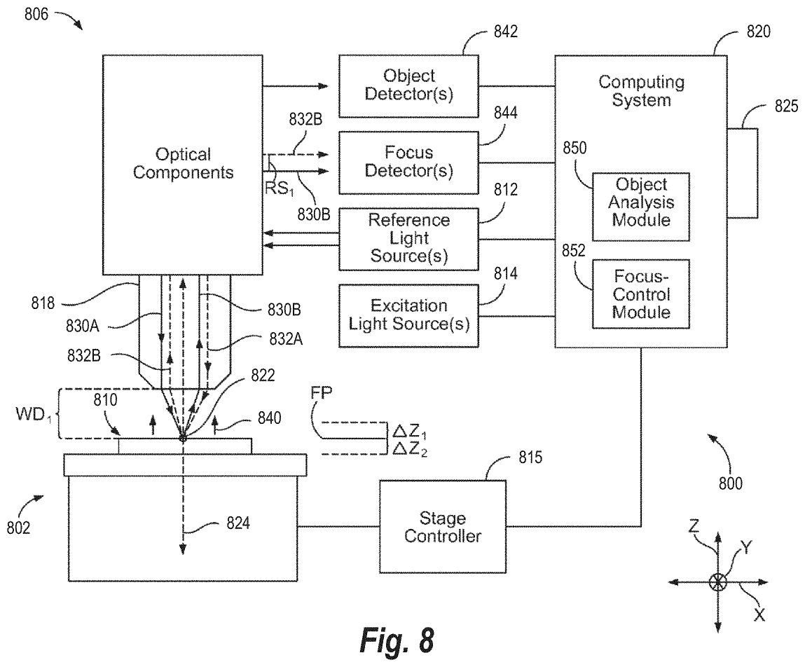

FIG. 8 illustrates a block diagram of an optical system 800 formed in accordance with one embodiment. By way of example only, the optical system 800 may be a sampler imager that images a sample of interest for analysis. In other embodiments, the optical system 800 may be a profilometer that determines a surface profile (e.g., topography) of an object. Furthermore, various other types of optical systems may use the mechanisms and systems described herein. In the illustrated embodiment, the optical system 800 includes an optical assembly 806, an object holder 802 for supporting an object 810 near a focal plane FP of the optical assembly 806, and a stage controller 815 that is configured to move the object holder 802 in a lateral direction (along an X-axis and/or a Y-axis that extend into the page) or in a vertical/elevational direction along a Z-axis. The optical system 800 may also include a system controller or computing system 820 that is operatively coupled to the optical assembly 806, the stage controller 815, and/or the object holder 802.

In particular embodiments, the optical system 800 is a sample imager configured to image samples. Although not shown, a sample imager may include other sub-systems or devices for performing various assay protocols. By way of example only, the sample may include a flow cell having flow channels. The sample imager may include a fluid control system that includes liquid reservoirs that are fluidically coupled to the flow channels through a fluidic network. The sample imager may also include a temperature control system that may have a heater/cooler configured to regulate a temperature of the sample and/or the fluid that flows through the sample. The temperature control system may include sensors that detect a temperature of the fluids.

As shown, the optical assembly 806 is configured to direct input light to an object 810 and receive and direct output light to one or more detectors. The output light may be input light that was at least one of reflected and refracted by the object 810 and/or the output light may be light emitted from the object 810. To direct the input light, the optical assembly 806 may include at least one reference light source 812 and at least one excitation light source 814 that direct light, such as light beams having predetermined wavelengths, through one or more optical components of the optical assembly 806. The optical assembly 806 may include various optical components, including a conjugate lens 818, for directing the input light toward the object 810 and directing the output light toward the detector(s).

In the exemplary embodiment, the reference light source 812 may be used by a distance measuring system or a focus-control system (or focusing mechanism) of the optical system 800 and the excitation light source 814 may be used to excite the biological or chemical substances of the object 810 when the object 810 includes a biological or chemical sample. The excitation light source 814 may be arranged to illuminate a bottom surface of the object 810, such as in TIRF imaging, or may be arranged to illuminate a top surface of the object 810, such as in epi-fluorescent imaging. As shown in FIG. 8, the conjugate lens 818 directs the input light to a focal region 822 lying within the focal plane FP. The lens 818 has an optical axis 824 and is positioned a working distance WD₁ away from the object 810 measured along the optical axis 824. The stage controller 815 may move the object 810 in the Z-direction to adjust the working distance WD₁ so that, for example, a portion of the object 810 is within the focal region 822.

To determine whether the object 810 is in focus (i.e., sufficiently within the focal region 822 or the focal plane FP), the optical assembly 806 is configured to direct at least one pair of light beams to the focal region 822 where the object 810 is approximately located. The object 810 reflects the light beams. More specifically, an exterior surface of the object 810 or an interface within the object 810 reflects the light beams. The reflected light beams then return to and propagate through the lens 818. As shown, each light beam has an optical path that includes a portion that has not yet been reflected by the object 810 and a portion that has been reflected by the object 810. The portions of the optical paths prior to reflection are designated as incident light beams 830A and 832A and are indicated with arrows pointing toward the object 810. The portions of the optical paths that have been reflected by the object 810 are designated as reflected light beams 830B and 832B and are indicated with arrows pointing away from the object 810. For illustrative purposes, the light beams 830A, 830B, 832A, and 832B are shown as having different optical paths within the lens 818 and near the object 810. However, in the exemplary embodiment, the light beams 830A and 832B propagate in opposite directions and are configured to have the same or substantially overlapping optical paths within the lens 818 and near the object 810, and the light beams 830B and 832A propagate in opposite directions and are configured to have the same or substantially overlapping optical paths within the lens 818 and near the object 810.

In the embodiment shown in FIG. 8, light beams 830A, 830B, 832A, and 832B pass through the same lens that is used for imaging. In an alternative embodiment, the light beams used for distance measurement or focus determination can pass through a different lens that is not used for imaging. In this alternative embodiment, the lens 818 is dedicated to passing beams 830A, 830B, 832A, and 832B for distance measurement or focus determination, and a separate lens (not shown) is used for imaging the object 810. Similarly, it will be understood that the systems and methods set forth herein for focus determination and distance measurement can occur using a common objective lens that is shared with the imaging optics or, alternatively, the objective lenses exemplified herein can be dedicated to focus determination or distance measurement.

The reflected light beams 830B and 832B propagate through the lens 818 and may, optionally, be further directed by other optical components of the optical assembly 806. As shown, the reflected light beams 830B and 832B are detected by at least one focus detector 844. In the illustrated embodiment, both reflected light beams 830B and 832B are detected by a single focus detector 844. The reflected light beams may be used to determine relative separation RS₁. For example, the relative separation RS₁ may be determined by the distance separating the beam spots from the impinging reflected light beams 830B and 832B on the focus detector 844 (i.e., a separation distance). The relative separation RS₁ may be used to determine a degree-of-focus of the optical system 800 with respect to the object 810. However, in alternative embodiments, each reflected light beam 830B and 832B may be detected by a separate corresponding focus detector 844 and the relative separation RS₁ may be determined based upon a location of the beam spots on the corresponding focus detectors 844.

If the object 810 is not within a sufficient degree-of-focus, the computing system 820 may operate the stage controller 815 to move the object holder 802 to a desired position. Alternatively or in addition to moving the object holder 802, the optical assembly 806 may be moved in the Z-direction and/or along the XY plane.

For example, the object 810 may be relatively moved a distance ΔZ₁ toward the focal plane FP if the object 810 is located above the focal plane FP (or focal region 822), or the object 810 may be relatively moved a distance ΔZ₂ toward the focal plane FP if the object 810 is located below the focal plane FP (or focal region 822). In some embodiments, the optical system 800 may substitute the lens 818 with another lens 818 or other optical components to move the focal region 822 of the optical assembly 806.

The example set forth above and in FIG. 8 has been presented with respect to a system for controlling focus or for determining degree-of-focus. The system is also useful for determining the working distance WD₁ between the object 810 and the lens 818. In such embodiments, the focus detector 844 can function as a working distance detector and the distance separating the beam spots on the working distance detector can be used to determine the working distance between the object 810 and the lens 818. For ease of description, various embodiments of the systems and methods are exemplified herein with regard to controlling focus or determining degree-of-focus. It will be understood that the systems and methods can also be used to determine the working distance between an object and a lens. Likewise, the systems and methods may also be used to determine a surface profile of an object.

In the exemplary embodiment, during operation, the excitation light source 814 directs input light (not shown) onto the object 810 to excite fluorescently-labeled biological or chemical substances. The labels of the biological or chemical substances provide light signals 840 (also called light emissions) having predetermined wavelength(s). The light signals 840 are received by the lens 818 and then directed by other optical components of the optical assembly 806 to at least one object detector 842. Although the illustrated embodiment only shows one object detector 842, the object detector 842 may comprise multiple detectors. For example, the object detector 842 may include a first detector configured to detect one or more wavelengths of light and a second detector configured to detect one or more different wavelengths of light. The optical assembly 806 may include a lens/filter assembly that directs different light signals along different optical paths toward the corresponding object detectors. Such optical systems are described in further detail by PCT Application No. PCT/US07/07991, entitled “System and Devices for Sequence by Synthesis Analysis”, filed Mar. 30, 2007 and PCT Application No. PCT/US2008/077850, entitled “Fluorescence Excitation and Detection System and Method”, filed Sep. 26, 2008, both of which the complete subject matter are incorporated herein by reference in their entirety.

The object detector 842 communicates object data relating to the detected light signals 840 to the computing system 820. The computing system 820 may then record, process, analyze, and/or communicate the data to other users or computing systems, including remote computing systems through a communication line (e.g., Internet). By way of example, the object data may include imaging data that is processed to generate an image(s) of the object 810. The images may then be analyzed by the computing system and/or a user of the optical system 800. In other embodiments, the object data may not only include light emissions from the biological or chemical substances, but may also include light that is at least one of reflected and refracted by the optical substrate or other components. For example, the light signals 840 may include light that has been reflected by encoded microparticles, such as the holographically encoded optical identification elements described above.

In some embodiments, a single detector may provide both functions as described above with respect to the object and focus detectors 842 and 844. For example, a single detector may detect the reflected light beams 830B and 832B and also the light signals 840.

The optical system 800 may include a user interface 825 that interacts with the user through the computing system 820. For example, the user interface 825 may include a display (not shown) that shows and requests information from a user and a user input device (not shown) to receive user inputs.

The computing system 820 may include, among other things, an object analysis module 850 and a focus-control module 852. The focus-control module 852 is configured to receive focus data obtained by the focus detector 844. The focus data may include signals representative of the beam spots incident upon the focus detector 844. The data may be processed to determine relative separation (e.g., separation distance between the beam spots). A degree-of-focus of the optical system 800 with respect to the object 810 may then be determined based upon the relative separation. In particular embodiments, the working distance WD₁ between the object 810 and lens 818 can be determined. Likewise, the object analysis module 850 may receive object data obtained by the object detectors 842. The object analysis module may process or analyze the object data to generate images of the object.

Furthermore, the computing system 820 may include any processor-based or microprocessor-based system, including systems using microcontrollers, reduced instruction set computers (RISC), application specific integrated circuits (ASICs), field programmable gate array (FPGAs), logic circuits, and any other circuit or processor capable of executing functions described herein. The above examples are exemplary only, and are thus not intended to limit in any way the definition and/or meaning of the term system controller. In the exemplary embodiment, the computing system 820 executes a set of instructions that are stored in one or more storage elements, memories, or modules in order to at least one of obtain and analyze object data. Storage elements may be in the form of information sources or physical memory elements within the optical system 800.

The set of instructions may include various commands that instruct the optical system 800 to perform specific protocols. For example, the set of instructions may include various commands for performing assays and imaging the object 810 or for determining a surface profile of the object 810. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

As described above, the excitation light source 814 generates an excitation light that is directed onto the object 810. The excitation light source 814 may generate one or more laser beams at one or more predetermined excitation wavelengths. The light may be moved in a raster pattern across portions of the object 810, such as groups in columns and rows of the object 810. Alternatively, the excitation light may illuminate one or more entire regions of the object 810 at one time and serially stop through the regions in a “step and shoot” scanning pattern. Line scanning can also be used as described, for example, in U.S. Pat. No. 7,329,860, of which the complete subject matter is incorporated herein by reference in its entirety. The object 810 produces the light signals 840, which may include light emissions generated in response to illumination of a label in the object 810 and/or light that has been reflected or refracted by an optical substrate of the object 810. Alternatively, the light signals 840 may be generated, without illumination, based entirely on emission properties of a material within the object 810 (e.g., a radioactive or chemiluminescent component in the object).

The object and focus detectors 842 and 844 may be, for example photodiodes or cameras. In some embodiments herein, the detectors 842 and 844 may comprise a camera that has a 1 mega pixel CCD-based optical imaging system such as a 1002×1004 CCD camera with 8 gm pixels, which at 20× magnification can optionally image an area of 0.4×0.4 mm per tile using an excitation light that has a laser spot size of 0.5×0.5 mm (e.g., a square spot, or a circle of 0.5 mm diameter, or an elliptical spot, etc.). Cameras can optionally have more or less than 1 million pixels, for example a 4 mega pixel camera can be used. In many embodiments, it is desired that the readout rate of the camera should be as fast as possible, for example the transfer rate can be 10 MHz or higher, for example 20 or 30 MHz. More pixels generally mean that a larger area of surface, and therefore more sequencing reactions or other optically detectable events, can be imaged simultaneously for a single exposure. In particular embodiments, the CCD camera/TIRF lasers may collect about 6400 images to interrogate 1600 tiles (since images are optionally done in 4 different colors per cycle using combinations of filters, dichroics and detectors as described herein). For a 1 Mega pixel CCD, certain images optionally can contain between about 5,000 to 50,000 randomly spaced unique nucleic acid clusters (i.e., images upon the flow cell surface). At an imaging rate of 2 seconds per tile for the four colors, and a density of 25000 clusters per tile, the systems herein can optionally quantify about 45 million features per hour. At a faster imaging rate, and higher cluster density, the imaging rate can be improved. For example, a readout rate of a 20 MHz camera, and a resolved cluster every 20 pixels, the readout can be 1 million clusters per second. A detector can be configured for Time Delay Integration (TDI) for example in line scanning embodiments as described, for example, in U.S. Pat. No. 7,329,860, of which the complete subject matter is incorporated herein by reference in its entirety. Other useful detectors include, but are not limited, to an optical quadrant photodiode detector, such as those having a 2×2 array of individual photodiode active areas fabricated on a single chip, examples of which are available from Pacific Silicon Sensor (Westlake Village, Calif.), or a position sensitive detector such as those having a monolithic PIN photodiode with a uniform resistance in one or two dimensions, examples of which are available from Hamamatsu Photonics, K.K., (Hamamatsu City, Japan).

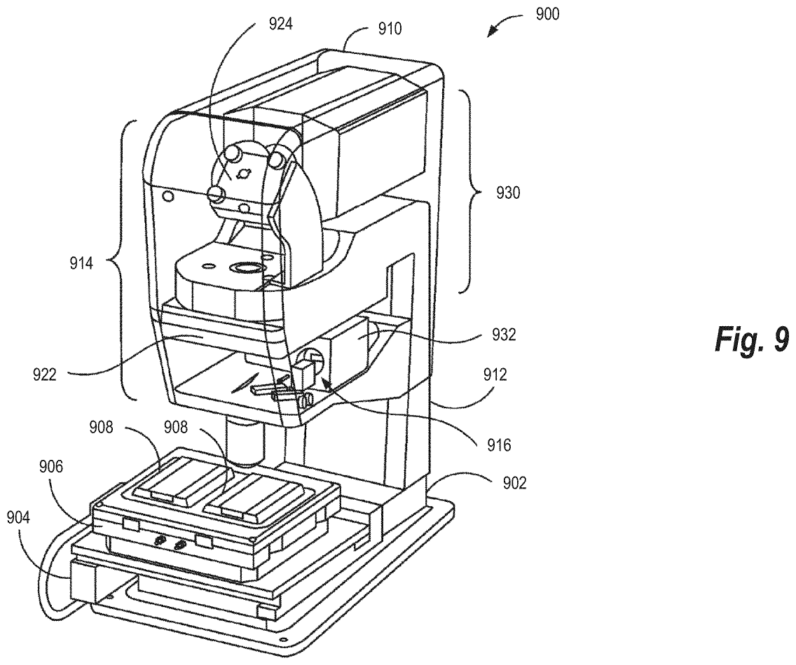

FIG. 9 is a perspective view of a sample imager 900 formed in accordance with one embodiment. As shown, the sample imager 900 includes an imager base 902 that supports a stage 904 having a sample holder 906 thereon. The sample holder 906 is configured to support one or more optical substrates 908 during an imaging session. The optical substrates 908 are illustrated as flow cells in FIG. 9. However, other samples may be used.

The sample imager 900 also includes a housing 910 (illustrated in phantom) and a strut 912 that supports the housing 910. The housing 910 can enclose at least a portion of an optical assembly 914 therein. The optical assembly 914 may include a focus assembly 916 and a sample-detecting assembly 930. For example, the focus assembly 916 may include an auto-focus line scan camera that receives reflected light beams for determining a degree-of-focus of the sampler imager 900. The sample imager 900 may also include a filter wheel 922 and an alignment mirror 924 that directs light toward a sample detector 932, which is shown as a K4 camera in FIG. 9.

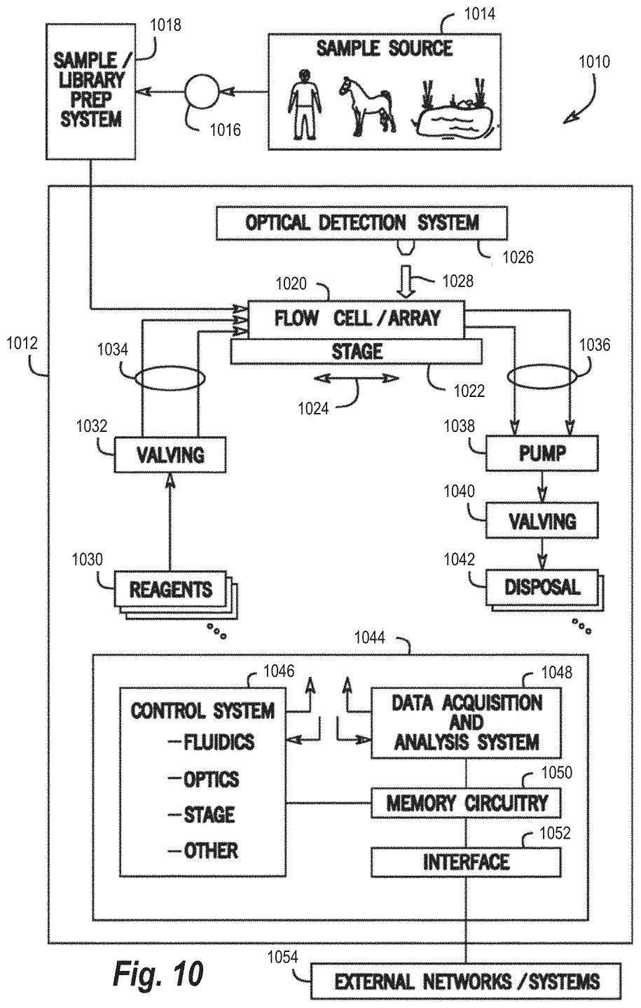

FIG. 10 illustrates an implementation of a sequencing system 1010 configured to process molecular samples that may be sequenced to determine their components, the component ordering, and generally the structure of the sample. The system includes an instrument 1012 that receives and processes a biological sample. A sample source 1014 provides the sample 1016 which in many cases will include a tissue sample. The sample source may include, for example, an individual or subject, such as a human, animal, microorganism, plant, or other donor (including environmental samples), or any other subject that includes organic molecules of interest, the sequence of which is to be determined. Of course, the system may be used with samples other than those taken from organisms, including synthesized molecules. In many cases, the molecules will include DNA, RNA, or other molecules having base pairs the sequence of which may define genes and variants having particular functions of ultimate interest.

The sample 1016 is introduced into a sample/library preparation system 1018. This system may isolate, break, and otherwise prepare the sample for analysis. The resulting library includes the molecules of interest in lengths that facilitate the sequencing operation. The resulting library is then provided to the instrument 1012 where the sequencing operation is performed. In practice, the library, which may sometimes be referred to as a template, is combined with reagents in an automated or semi-automated process, and then introduced to the flow cell prior to sequencing.

In the implementation illustrated in FIG. 10, the instrument includes a flow cell or array 1020 that receives the sample library. The flow cell includes one or more fluidic channels that allow for sequencing chemistry to occur, including attachment of the molecules of the library, and amplification at locations or sites that can be detected during the sequencing operation. For example, the flow cell/array 1020 may include sequencing templates immobilized on one or more surfaces at the locations or sites. A “flow cell” may include a patterned array, such as a microarray, a nanoarray, and so forth. In practice, the locations or sites may be disposed in a regular, repeating pattern, a complex non-repeating pattern, or in a random arrangement on one or more surfaces of a support. To enable the sequencing chemistry to occur, the flow cell also allows for introduction of substances, such as including various reagents, buffers, and other reaction media, that are used for reactions, flushing, and so forth. The substances flow through the flow cell and may contact the molecules of interest at the individual sites.

In the instrument the flow cell 1020 is mounted on a movable stage 1022 that, in this implementation, may be moved in one or more directions as indicated by reference numeral 1024. The flow cell 1020 may, for example, be provided in the form of a removable and replaceable cartridge that may interface with ports on the movable stage 1022 or other components of the system in order to allow reagents and other fluids to be delivered to or from the flow cell 1020. The stage is associated with an optical detection system 1026 that can direct radiation or light 1028 to the flow cell during sequencing. The optical detection system may employ various methods, such as fluorescence microscopy methods, for detection of the analytes disposed at the sites of the flow cell. By way of non-limiting example, the optical detection system 1026 may employ confocal line scanning to produce progressive pixilated image data that can be analyzed to locate individual sites in the flow cell and to determine the type of nucleotide that was most recently attached or bound to each site. Other imaging techniques may also suitably be employed, such as techniques in which one or more points of radiation are scanned along the sample or techniques employing “step and shoot” imaging approaches. The optical detection system 1026 and the stage 1022 may cooperate to maintain the flow cell and detection system in a static relationship while obtaining an area image, or, as noted, the flow cell may be scanned in any suitable mode (e.g., point scanning, line scanning, “step-and-shoot” scanning).

While many different technologies may be used for imaging, or more generally for detecting the molecules at the sites, presently contemplated implementations may make use of confocal optical imaging at wavelengths that cause excitation of fluorescent tags. The tags, excited by virtue of their absorption spectrum, return fluorescent signals by virtue of their emission spectrum. The optical detection system 1026 is configured to capture such signals, to process pixelated image data at a resolution that allows for analysis of the signal-emitting sites, and to process and store the resulting image data (or data derived from it).

In a sequencing operation, cyclic operations or processes are implemented in an automated or semi-automated fashion in which reactions are promoted, such as with single nucleotides or with oligonucleotides, followed by flushing, imaging and de-blocking in preparation for a subsequent cycle. The sample library, prepared for sequencing and immobilized on the flow cell, may undergo a number of such cycles before all useful information is extracted from the library. The optical detection system 1026 may generate image data from scans of the flow cell (and its sites) during each cycle of the sequencing operation by use of electronic detection circuits (e.g., cameras or imaging electronic circuits or chips). The resulting image data may then be analyzed to locate individual sites in the image data, and to analyze and characterize the molecules present at the sites, such as by reference to a specific color or wavelength of light (a characteristic emission spectrum of a particular fluorescent tag) that was detected at a specific location, as indicated by a group or cluster of pixels in the image data at the location. In a DNA or RNA sequencing application, for example, the four common nucleotides may be represented by distinguishable fluorescence emission spectra (wavelengths or wavelength ranges of light). Each emission spectrum, then, may be assigned a value corresponding to that nucleotide. Based upon this analysis, and tracking the cyclical values determined for each site, individual nucleotides and their orders may be determined for each site. These sequences may then be further processed to assemble longer segments including genes, chromosomes, and so forth. As used in this disclosure the terms “automated” and “semi-automated” mean that the operations are performed by system programming or configuration with little or no human interaction once the operations are initiated, or once processes including the operations are initiated.

In the illustrated implementation, reagents 1030 are drawn or aspirated into the flow cell through valving 1032. The valving may access the reagents from recipients or vessels in which they are stored, such as through pipettes or sippers (not shown in FIG. 10). The valving 1032 may allow for selection of the reagents based upon a prescribed sequence of operations performed. The valving may further receive commands for directing the reagents through flow paths 1034 into the flow cell 1020. Exit or effluent flow paths 1036 direct the used reagents from the flow cell. In the illustrated implementation, a pump 1038 serves to move the reagents through the system. The pump may also serve other useful functions, such as measuring reagents or other fluids through the system, aspirating air or other fluids, and so forth. Additional valving 1040 downstream of pump 1038 allows for appropriately directing the used reagent to disposal vessels or recipients 1042.

The instrument further includes a range of circuitry that aids in commanding the operation of the various system components, monitoring their operation by feedback from sensors, collecting image data, and at least partially processing the image data. In the implementation illustrated in FIG. 10, a control/supervisory system 1044 includes a control system 1046 and a data acquisition and analysis system 1048. Both systems will include one or more processors (e.g., digital processing circuits, such as microprocessors, multi-core processors, FPGA's, or any other suitable processing circuitry) and associated memory circuitry 1050 (e.g., solid state memory devices, dynamic memory devices, on and/or off-board memory devices, and so forth) that may store machine-executable instructions for controlling, for example, one or more computers, processors, or other similar logical devices to provide certain functionality. Application-specific or general purpose computers may at least partially make up the control system and the data acquisition and analysis system. The control system may include, for example, circuitry configured (e.g., programmed) to process commands for fluidics, optics, stage control, and any other useful functions of the instrument. The data acquisition and analysis system 1048 interfaces with the optical detection system to command movement of the optical detection system or the stage, or both, the emission of light for cyclic detection, receiving and processing of returned signals, and so forth. The instrument may also include various interfaces as indicated at reference 1052, such as an operator interface that permits control and monitoring of the instrument, transfer of samples, launching of automated or semi-automated sequencing operations, generation of reports, and so forth. Finally, in the implementation of FIG. 10, external networks or systems 1054 may be coupled to and cooperate with the instrument, for example, for analysis, control, monitoring, servicing, and other operations.

It may be noted that while a single flow cell and fluidics path, and a single optical detection system 1026 are illustrated in FIG. 10, in some instruments more than one flow cell and fluidics path may be accommodated. For example, in a presently contemplated implementation, two such arrangements are provided to enhance sequencing and throughput. In practice, any number of flow cells and paths may be provided. These may make use of the same or different reagent receptacles, disposal receptacles, control systems, image analysis systems, and so forth. Where provided, the multiple fluidics systems may be individually controlled or controlled in a coordinated fashion.

We claim:

1. A system comprising:
a server corresponding to a sequencing device; and
a non-transitory computer readable medium comprising instructions that, when executed by at least one processor of the server, cause the system to:
receive, from a computing device, a status query for a nucleotide sequencing taskset depicted by an active sequencing interface;
determine, for the nucleotide sequencing taskset, statuses of a sequencing run, a data-analysis transfer of base-call data generated during the sequencing run, and a variant analysis of the base-call data; and
provide, for display within the active sequencing interface, a graphical status summary for the nucleotide sequencing taskset comprising a run status icon indicating a status of the sequencing run, a data-transfer-status icon indicating a status of the data-analysis transfer, and a variant-analysis-status icon indicating a status of the variant analysis.

2. The system of claim 1, further comprising instructions that, when executed by the at least one processor, cause the system to:
determine a collective base-call-quality metric indicating an accuracy of base calls generated during the sequencing run; and
provide, for display within the active sequencing interface, the collective base-call-quality metric.

3. The system of claim 1, further comprising instructions that, when executed by the at least one processor, cause the system to:
determine a collective pass filter metric indicating a subset of base calls generated during the sequencing run that satisfy a quality filter; and
provide, for display within the active sequencing interface, the collective pass filter metric.

4. The system of claim 1, further comprising instructions that, when executed by the at least one processor, cause the system to:
determine an updated status of one or more of the sequencing run, the data-analysis transfer, or the variant analysis; and
provide an updated graphical status summary comprising one or more of an updated run status icon indicating an updated status of the sequencing run, an updated data-transfer-status icon indicating an updated status of the data-analysis transfer, and an updated variant-analysis-status icon indicating an updated status of the variant analysis.

5. The system of claim 1, further comprising instructions that, when executed by the at least one processor, cause the system to:
determine a status of an external-call-data transfer of the base-call data generated during the sequencing run to an external storage; and
provide the graphical status summary further comprising an external-transfer-status icon indicating a status of the external-call-data transfer.

6. The system of claim 1, further comprising instructions that, when executed by the at least one processor, cause the system to:
determine a status of an external-analysis-data transfer of data generated during the variant analysis to an external storage; and
provide the graphical status summary further comprising an external-transfer-status icon indicating a status of the external-analysis-data transfer.

7. The system of claim 1, further comprising instructions that, when executed by the at least one processor, cause the system to provide the graphical status summary for display on the sequencing device, local computing device connected to a local server, or an external computing device connected to the server via a network.

8. A non-transitory computer readable storage medium comprising instructions that, when executed by at least one processor, cause a system to:
receive, from a computing device, a status query for a nucleotide sequencing taskset depicted by an active sequencing interface;
determine, for the nucleotide sequencing taskset and from a sequencing device, a status of a sequencing run;
determine, for the nucleotide sequencing taskset and from a local server corresponding to the sequencing device, statuses of a data-analysis transfer of base-call data generated during the sequencing run and a variant analysis of the base-call data; and
provide, for display within the active sequencing interface, a graphical status summary for the nucleotide sequencing taskset comprising a run status icon indicating a status of the sequencing run, a data-transfer-status icon indicating a status of the data-analysis transfer, and a variant-analysis-status icon indicating a status of the variant analysis.

9. The non-transitory computer readable storage medium of claim 8, further comprising instructions that, when executed by the at least one processor, cause the system to determine the status of the data-analysis transfer by determining a status of a transfer of base-call data generated during the sequencing run from the sequencing device to the local server.

10. The non-transitory computer readable storage medium of claim 8, further comprising instructions that, when executed by the at least one processor, cause the system to:
receive an indication of a user selection of an expand option corresponding to the graphical status summary; and
based on the indication of the user selection of the expand option, provide, for display within the active sequencing interface, an expanded graphical status summary comprising a textual status summary for the run status icon, a textual status summary for the data-transfer-status icon, and a textual status summary for the variant-analysis-status icon.

11. The non-transitory computer readable storage medium of claim 8, further comprising instructions that, when executed by the at least one processor, cause the system to:
receive an indication of a user selection of a cancel option corresponding to one or more of the sequencing run, the data-analysis transfer, or the variant analysis; and
based on the indication of the user selection of the cancel option, cancel one or more of the sequencing run, the data-analysis transfer, or the variant analysis.

12. The non-transitory computer readable storage medium of claim 8, further comprising instructions that, when executed by the at least one processor, cause the system to:
receive an indication of a user selection of a re-initiate option corresponding to one or more of the sequencing run, the data-analysis transfer, or the variant analysis; and
based on the indication of the user selection of the re-initiate option, re-initiate one or more of the sequencing run, the data-analysis transfer, or the variant analysis.

13. The non-transitory computer readable storage medium of claim 8, further comprising instructions that, when executed by the at least one processor, cause the system to:
determine a collective base-call-quality metric indicating an accuracy of base calls generated during the sequencing run; and
provide, for display within the active sequencing interface, the collective base-call-quality metric proximate to the graphical status summary.

14. The non-transitory computer readable storage medium of claim 8, further comprising instructions that, when executed by the at least one processor, cause the system to:
determine a collective pass filter metric indicating a subset of base calls generated during the sequencing run that satisfy a quality filter; and
provide, for display within the active sequencing interface, the collective pass filter metric proximate to the graphical status summary.

15. A method comprising:
receiving, from a computing device, one or more status queries for nucleotide sequencing tasksets depicted by an active sequencing interface;
determining, for the nucleotide sequencing tasksets, statuses of respective sequencing runs, respective data-analysis transfers of base-call data generated during the respective sequencing runs, and respective variant analyses of the base-call data; and
providing, for display within the active sequencing interface, graphical status summaries for the nucleotide sequencing tasksets comprising run status icon indicating statuses of the respective sequencing runs, data-transfer-status icons indicating statuses of the respective data-analysis transfers, and variant-analysis-status icons indicating statuses of the respective variant analyses.

16. The method of claim 15, wherein determining the statuses comprises:
determining, for a first nucleotide sequencing taskset among the nucleotide sequencing tasksets, statuses of a first sequencing run, a first data-analysis transfer of base-call data generated during the first sequencing run, and a first variant analysis of the base-call data; and
determining, for a second nucleotide sequencing taskset among the nucleotide sequencing tasksets, statuses of a second sequencing run, a second data-analysis transfer of base-call data generated during the second sequencing run, and a second variant analysis of the base-call data.

17. The method of claim 16, wherein providing the graphical status summaries for the nucleotide sequencing tasksets comprises providing, for display within the active sequencing interface:
a first graphical status summary for the first nucleotide sequencing taskset comprising a first run status icon indicating a status of the first sequencing run, a first data-transfer-status icon indicating a status of the first data-analysis transfer, and a first variant-analysis-status icon indicating a status of the first variant analysis; and
a second graphical status summary for the second nucleotide sequencing taskset comprising a second run status icon indicating a status of the second sequencing run, a second data-transfer-status icon indicating a status of the second data-analysis transfer, and a second variant-analysis-status icon indicating a status of the second variant analysis.

18. The method of claim 15, further comprising:
determining statuses of respective external-call-data transfers of the base-call data generated during the respective sequencing runs to an external storage; and
providing the graphical status summaries further comprising external-transfer-status icons indicating statuses of the respective external-call-data transfers.

19. The method of claim 15, further comprising:
determining statuses of respective external-analysis-data transfers of data generated during the respective variant analyses to an external storage; and
providing the graphical status summaries further comprising external-transfer-status icons indicating statuses of the respective external-analysis-data transfers.

20. The method of claim 15, further comprising:
receiving an indication of a user selection of an expand option corresponding to a graphical status summary among the graphical status summaries; and
based on the indication of the user selection of the expand option, provide, for display within the active sequencing interface, an expanded graphical status summary comprising a textual status summary for a run status icon within the graphical status summary, a textual status summary for a data-transfer-status icon within the graphical status summary, and a textual status summary for a variant-analysis-status icon within the graphical status summary.
